(19) United States
(12) Patent Application Publication
(10) Pub. No.: US 2025/0178999 A1
(43) Pub. Date: Jun. 5, 2025

(54) ONE STEP SYNTHESIS OF 1-TETRALONE COMPOUNDS AND USES THEREOF IN THE PREPARATION OF (+)-TETRALONE ABSCISIC ACID (ABA)

(71) Applicant: University of Saskatchewan, Saskatoon (CA)

(72) Inventors: Naveen Diddi, Saskatchewan (CA); Suzanne Roberta Abrams, Saskatoon (CA); Leon Lai, Saskatoon (CA)

(73) Assignee: University of Saskatchewan, Saskatchewan, SK (CA)

(21) Appl. No.: 18/844,118

(22) PCT Filed: Mar. 16, 2023
(86) PCT No.: PCT/CA2023/050338

§ 371 Date: Sep. 5, 2024

Related U.S. Application Data
(60) Provisional application No. 63/321,316, filed on Mar. 18, 2022.

(51) Int. Cl. C07C 51/353

(52) U.S. Cl. CPC C07C 51/353

(57) ABSTRACT

The present application is related to a process for preparing 1-tetralone compounds such as compounds of Formula I by reacting a,|3-unsaturated cyclic ketones that comprise two available hydrogens on the y carbon to the carbonyl with acrolein derivatives with heating in the presence of a suitable organic acid, a suitable organic amine base and suitable inert solvent. For example, the 1-tetralone compound is a tetralone derivative of abscisic acid (ABA).

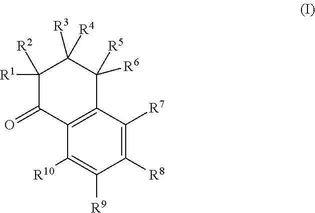

### CROSS-REFERENCE TO RELATED APPLICATIONS

The present application claims the benefit of priority from U.S. provisional patent application No. 63/321,316 filed on Mar. 18, 2022, the contents of which are incorporated herein by reference in their entirety.

### FIELD

The present application is related to process for preparing 1-tetralone compounds. In particular the process involves the reaction of acrolein derivatives with α,β-unsaturated cyclic ketones that comprise two available hydrogens on the γ carbon relative to the alpha, beta-unsaturated ketone.

### BACKGROUND

Climate change and global warming are resulting in plants experiencing increased environmental stress which negatively affects agricultural productivity (Raza et al 2019). Plant breeding as well as biotechnological and chemical approaches are being employed towards improving plants' resistance to environmental stresses such as heat, cold and drought. To this end, a number of multidisciplinary teams are focusing on developing plant growth regulators based on the plant hormone abscisic acid (ABA, 1, Scheme 1), a carotenoid-derived sesquiterpenoid present in all plants. ABA triggers plant responses to extremes of temperature and drought, and plays numerous roles over a plant's life including seed development, dormancy and secondary metabolite production (Helander et al 2016; Gupta et al 2020; Cutler et al 2010; Vaidya et al 2021; Frankenpohl et al 2018; Yoshida et al 2021).

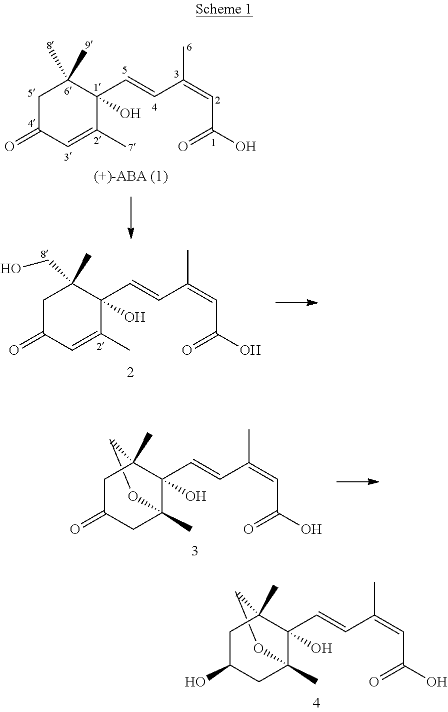

The natural product (S)-ABA (1), is produced on large scale by fermentation and is readily available (Rademacher 2015). It is commercially used for several applications including enhancing color of red table grapes, as a seed treatment for hybrid seed production and for increasing shelf life and transplanting of bedding plants and vegetable seedlings. Uses of ABA are limited by its short half-life in plants and toxicity at high concentrations. The level of ABA in plants is regulated principally by hydroxylation of the 8′-methyl group to generate 8′-hydroxy ABA (2) that is in equilibrium with the cyclized form phaseic acid (PA, 3), which is formed by 1,4-conjugate addition of the 8′-oxygen to the 2′-carbon of 2 (Scheme 1). Reduction of the ketone of 3 affords the metabolite dihydrophaseic acid (DPA, 4). Both PA 3 and DPA 4 are generally considered to be inactive catabolites of ABA (Abrams and Loewen 2019).

Numerous structural analogs of ABA targeting the binding pocket of receptors have been synthesized as potential receptor agonists as growth regulating chemicals that have greater potency and stability in vivo (Gupta et al 2020). The tetralone ABA analog 5 (I-1) and the 8′-acetylene ABA analog 6 were designed to replace ABA in the binding site of the receptor and have longer lasting effects than ABA itself (Abrams and Loewen 2019) (Scheme 2). Recently, some very active agonists such as opabactin (OP, 7) have been reported that bind at sites of ABA receptors other than the ABA binding pocket (Vaidya et al 2019).

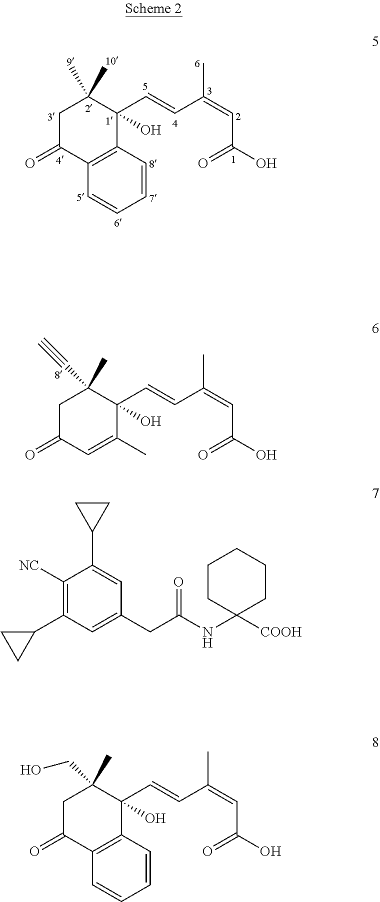

In a corn cell suspension system, the tetralone ABA analog 5 was found to be oxidized to the hydroxylated metabolite 8 which was not converted to a phaseic acid-like compound due to the aromatic ring fused to the ring of ABA. Both the tetralone 5 and 8 were found to retain biological activity (Nyangulu et al 2006).

The tetralone ABA analog 5 has been tested and compared to ABA in plant assays and in all cases, found to have activity equal to or stronger than ABA itself (Nyangulu et al 2006; Han et al 2015; Benson et al 2015; Gordon et al 2016; Vaidya et al 2019; Wan et al 2019). In an initial literature report describing the synthesis of the tetralone ABA 5, the (+)-enantiomer was reported to have higher activity than ABA in both a corn cell growth inhibition assay as well as a seed germination inhibition assay in Arabidopsis (Nyangulu et al 2006). Han et al 2015 reported that the racemic tetralone ABA (±)-5 had higher activity than racemic ABA ((±)-1 in seed germination assays in arabidopsis, lettuce and in a rice seedling growth inhibition assay. In a wide screen of a series of structural analogs of ABA in both physiological and receptor assays in Arabidopsis, Benson et al 2015 reported that the tetralone ABA (+)-5 had similar effects to (+)-1 in germination, root growth, and stomatal assays in Arabidopsis, as well as in receptor assays for AB11, PP2CA and HAB1 activity regulated by RCAR1. Gordon et al 2016 reported similar results in receptor assays in wheat. More recently, the tetralone ABA analog (+)-5 was tested along with compounds structurally unrelated to ABA, particularly opabactin 7 (Scheme 2) in receptor-based assays in Arabidopsis and in wheat. The tetralone (+)-5 (IC₅₀ 194+/−16 nm) displayed similar or greater activity than (+)-1 (IC₅₀ 601+/−14 nm) and weaker than 7 (IC₅₀ 62+/−3 nm) in germination of Arabidopsis (Vaidya et al 2019). In receptor-based assays in Arabidopsis and wheat, 7 was more the active in subgroup III, while (+)-5 was more potent in subgroup II.

The first synthesis of racemic methyl ester of tetralone ABA (±)-I-2 was achieved from 1-tetralone in a six-step linear sequence (installation of the geminal methyl groups, addition of dilithium salt of cis-3-methyl-2-penten-4-yn-1-ol to the ketone, conversion of the sidechain to the 2-cis-4-trans dienoic ester, oxidation of the benzylic carbon either before or after sidechain addition) with 15% overall yield (Scheme 3) (Nyangulu et al 2006). The desired (+)-enantiomer ((+)-5, I-1) of the resulting racemic tetralone methyl ester (±)-I-2 was separated by HPLC using a chiral column and was subjected to hydrolysis to afford (+)-5 in milligram quantities (Nyangulu et al 2006; Han et al 2015). This synthesis, while successful, is laborious, time consuming and not suitable for producing multigram quantities of tetralone ABA (+)-5 needed for field studies and further development as, for example, a practical plant growth regulator.

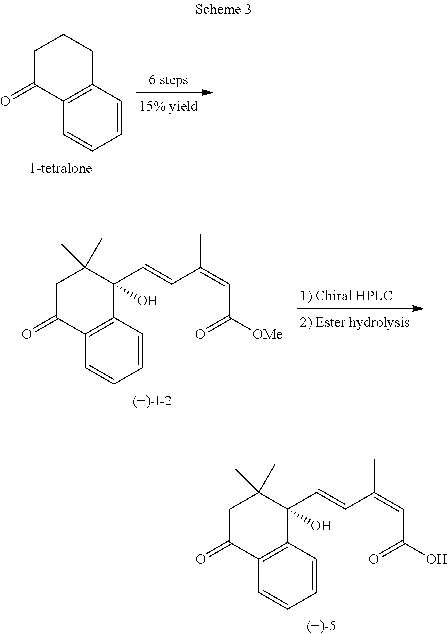

Isophorone has been converted to its tetralone derivatives 9 and 10 using vinylogous amide acetals and aminal esters (Jutz 1975) or with vinylogous amidinium salts (Bredereck et 1965; Bredereck et al 1968) (Scheme 4). This transformation was highly regioselective, in that the condensation occurred exclusively at the carbon atom gamma to the carbonyl, and no reaction at the α-methylene group to the carbonyl was reported.

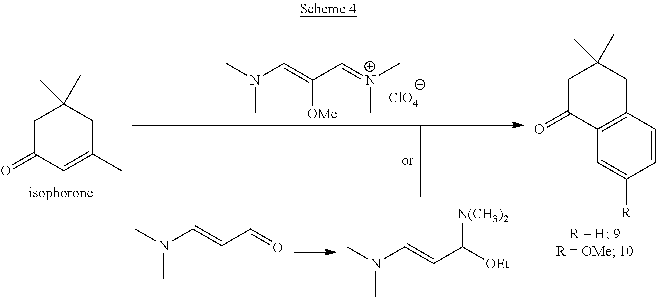

Schröder (Schröder 1990) reported that a Knoevenagel condensation between 3-dimethylaminoacrolein and ethyl cyanoacetate (or a substrate with an active methylene group) in presence of acetic acid and piperidine produced the α,β-unsaturated ester 11 as an intermediate that was used in synthesis of 2-chloronicotinic acid (Scheme 5).

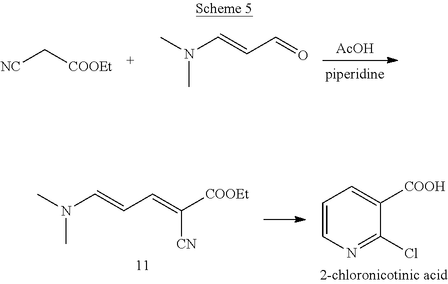

### SUMMARY

The present application includes a process for preparing a compound of Formula (I):

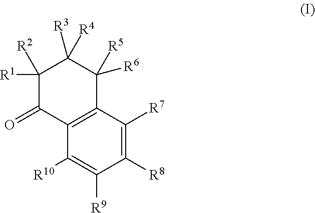

comprising reacting a compound of Formula (II)

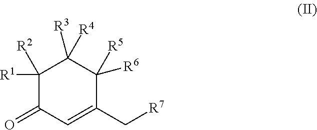

with a compound of Formula (III),

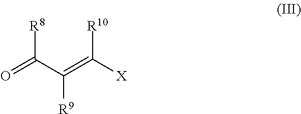

in the presence of a suitable organic acid, suitable organic amine base and a suitable inert solvent, with heating under conditions to remove water,
wherein
R¹, R², R³, R⁴, R⁵ and R⁶ are independently selected from H, halo, OH, CN, C₁₋₃alkyl, C₂₋₄alkenyl, C₂₋₄alkynyl, C₃₋₆cycloalkyl, C₆₋₁₀aryl and C₁₋₃alkyl substituted with one or more OH; wherein each cycloalkyl and aryl optionally substituted with one or more substituents independently selected from OH, halo, C₁₋₆alkyl and OC₁₋₆alkyl;
one of R¹, R², R³, R⁴, R⁵ and R⁶ is further optionally selected from C₃₋₁₀alkyl, C₄₋₁₀alkenyl, C₄₋₁₀alkynyl, ZC₁₋₁₀alkylene, ZC₂₋₁₀alkenylene, ZC₂₋₁₀alkynylene, C₁₋₁₀alkyleneZ′, C₂₋₁₀alkenyleneZ′, and C₂₋₄alkynyleneZ′, wherein each alkyl, alkenyl, alkynyl, alkylene, alkenylene and alkynylene is optionally substituted with one or more substituents independently selected from OH, halo, C₁₋₆alkyl and OC₁₋₆alkyl;
each Z is independently selected from O, S, C(O), S(O), SO₂, NR¹¹, NR¹¹C(O) and C(O)NR¹¹;
each Z′ is independently selected from OR¹², CO₂R¹³, C(O)R¹², NR¹²R¹⁴; and C(O)NR¹²R¹⁴;
X is selected from halo, OR¹⁵ and NR¹⁵R¹⁶;
R⁷ is selected from H, halo, OR¹⁷, C(O)R¹⁷, C₁₋₁₀alkyl and C₁₋₁₀alkyl substituted with one or more OH;
R⁸, R⁹ and R¹⁰ are independently selected from H, C₁₋₁₀alkyl, OC₁₋₁₀alkyl, C₆₋₁₀aryl, and C₆₋₁₀aryl optionally substituted with one or more substituents independently selected from OH, halo, C₁₋₆alkyl and OC₁₋₆alkyl;
R¹¹, R¹², R¹⁴, R¹⁵, R¹⁶ and R¹⁷ are independently selected from H and C₁₋₁₀alkyl; and
R¹³ is C₁₋₁₀alkyl,
provided at least two of R¹, R², R³ and R⁴ are other than H.

In some embodiments, the present application includes a process of process for preparing a compound of Formula (I) wherein the compound of Formula II is the methyl ester of (+)-ABA (II-1):

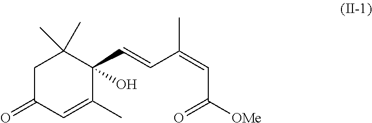 [SMILES: COC(=O)/C=C(C)\C=C\[C@@]1(O)C(C)=CC(=O)CC1(C)C]

and the compound of Formula III is 3-dimethylaminoacrolein (III-1):

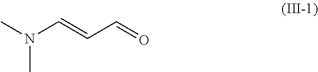

and the process of the application provides the (+)-ABA tetralone methyl ester of Formula I-1:

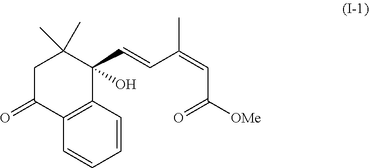 [SMILES: COC(=O)/C=C(C)\C=C\[C@@]1(O)c2ccccc2C(=O)CC1(C)C]

The present application further includes a process for preparing (+)-ABA tetralone (i.e. (+)-5):

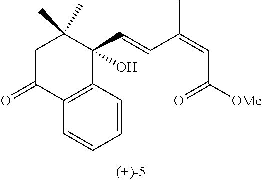

comprising hydrolyzing the compound of Formula I-1, prepared by the process of the application.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DETAILED DESCRIPTION

### I. Definitions

Unless otherwise indicated, the definitions and embodiments described in this, and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

The term “process of the application” and the like as used herein refers to a process of preparing 1-tetralone compounds including compounds of Formula (I) or (I-A) as described herein.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

As used in the present application, the singular forms “a”, “an” and “the” include plural references unless the content clearly dictates otherwise. For example, an embodiment including “a solvent” should be understood to present certain aspects with one solvent, or two or more additional solvents.

In embodiments comprising an “additional” or “second” component, such as an additional or second solvent, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms. All alkyl groups are optionally fluoro-substituted.

The term “alkenyl” as used herein, whether it is used alone or as part of another group, means straight or branched chain, unsaturated alkyl groups containing at least one double bond. The number of carbon atoms that are possible in the referenced alkyl group are indicated by the prefix “Cₙ₁₋ₙ₂”. For example, the term C₂₋₆alkenyl means an alkenyl group having 2, 3, 4, 5 or 6 carbon atoms. All alkenyl groups are optionally fluoro-substituted.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms. All alkynyl groups are optionally fluoro-substituted.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms. All alkylene groups are optionally fluoro-substituted.

The term “alkenylene” as used herein, whether it is used alone or as part of another group, means a straight or branched chain, unsaturated alkylene group, that is, an unsaturated carbon chain that contains substituents on two of its ends and at least one double bond. The number of carbon atoms that are possible in the referenced alkenylene group are indicated by the prefix “Cₙ₁₋ₙ₂”. For example, the term C₂₋₆alkenylene means an alkenylene group having 2, 3, 4, 5 or 6 carbon atoms. All alkenylene groups are optionally fluorosubstituted.

The term “alkynylene” as used herein, whether it is used alone or as part of another group, means a straight or branched chain, unsaturated alkylene group, that is, an unsaturated carbon chain that contains substituents on two of its ends and at least one triple bond. The number of carbon atoms that are possible in the referenced alkynylene group are indicated by the prefix “Cₙ₁₋ₙ₂”. For example, the term C₂₋₆alkynylene means an alkynylene group having 2, 3, 4, 5 or 6 carbon atoms. All alkynylene groups are optionally fluorosubstituted.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains from 6 to 10 carbon atoms.

All cyclic groups, including aryl and cycloalkyl groups, contain one (i.e. are monocyclic) or more than one ring (i.e. are polycyclic). When a cyclic group contains more than one ring, the rings may be fused, bridged or spirofused.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “unsubstituted”, as used herein means that the referenced atom does not contain a substituent group other than a hydrogen atom.

The term “substituted” as used herein means that the referenced atom contains at least one substituent group other that a hydrogen atom.

The term “substituent group” as used herein refers to any chemical grouping, including groups comprising carbon atoms and/or heteroatoms) that is compatible with the reaction conditions of the processes of the application.

The term “major isomer” as used herein refers to a stereochemical isomer, including a regional isomer, that is the most abundant isomer in a mixture of isomers of the same compound. Conversely, the term “minor isomer” as used herein refers to a stereochemical isomer, including a regional isomer, that is not the most abundant isomer in a mixture of isomers of the same compound.

In the processes of the application, it is typical for the compounds, including starting materials and products to be present as a mixture of isomers. For example, when it is shown that the R- or S-isomer is a product or starting material of a reaction, this means that that isomer is present in greater than 80%, 85%, 90%, 95%, 98% or 99% by weight based on the total amount of R- and S-isomers.

The products of the processes of the application may be isolated according to known methods, for example, the compounds may be isolated by evaporation of the solvent, by filtration, centrifugation, chromatography or other suitable method.

The term “azeotrope” as used herein refers to a mixture of two or more solvents that has a constant boiling point. The components of an azeotrope cannot be separated via simple distillation. An azeotrope may be characterized as a positive azeotrope (e.g., a mixture having a lower boiling point than either of its components) or a negative azeotrope (e.g., a mixture having a higher boiling point than either of its components).

The symbol [symbol] when drawn perpendicularly across a bond indicates a point of covalent attachment of a chemical group

The term “abscisic acid”, “(+)-ABA” or “(S)-ABA” refers to a compound having the chemical name: (2Z,4E)-5-[(1S)-1-hydroxy-2,6,6-trimethyl-4-oxocyclohex-2-en-1-yl]-3-methylpenta-2,4-dienoic acid and having the chemical formula:

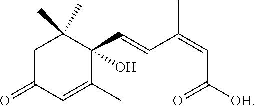

The term “(+)-ABA tetralone” or compound (+)-5 refers to a compound having the chemical name: (2Z,4E)-5-((S)-1-hydroxy-2,2-dimethyl-4-oxo-1,2,3,4-tetrahydronaphthalen-1-yl)-3-methylpenta-2,4-dienoic acid and having the chemical formula:

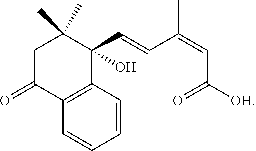

The term “racemic ABA tetralone” refers to a compound having the chemical name: ((2Z,4E)-5-(1-hydroxy-2,2-dimethyl-4-oxo-1,2,3,4-tetrahydronaphthalen-1-yl)penta-2,4-dienoic acid and having the chemical formula:

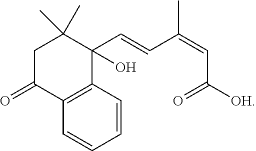 [SMILES: CC(=C/C(=O)O)/C=C/C1(O)c2ccccc2C(=O)CC1(C)C]

### II. Processes of the Application

The Applicants have developed an efficient, high yielding process for preparing 1-tetralone compounds from α,β-unsaturated cyclic ketones that comprise two available hydrogens on the γ carbon to the carbonyl. More specifically, a general process for preparing 1-tetralone compounds through a condensation cyclization reaction between acrolein derivatives and α,β-unsaturated cyclic ketones that comprise two available hydrogens on the γ carbon to the carbonyl, in the presence of a suitable organic acid, organic amine base and inert solvents with heating has been developed.

Further, the Applicants have found that the process of the application can be used in a two step high yielding efficient synthesis of natural compounds of interest, such as in the synthesis of enantiopure tetralone ABA (+)-5 from the ester derivative of the readily available α,β-unsaturated cyclic ketone, (S)-ABA (1).

Initial attempts to form the 1-tetralone ring of ABA (+)-5 from the known 7′-aldehyde of the ABA methyl ester, (Shimomura et al. 2007), either by Grignard reaction with allyl bromide, or by Wittig reaction with the ylid of allyl triphenyl phosphonium bromide resulted in poor yields. A vinylogous aldol reaction of ABA methyl ester (II-1) with benzaldehyde derivatives was reported to give products substituted at the 7′-carbon atom (Wang et al. 2016). A similar vinylogous aldol reaction of ABA methyl ester (II-1) with crotonaldehyde was found to afford the corresponding 7′-aldol adduct in reasonable yield. However, the 7′-aldol adduct did not undergo further dehydration or cyclization using various reaction conditions. Further, attempted condensation reactions of isophorone or ABA methyl ester (II-1) with the known dimethyl sulfate and perchlorate adducts of 3-dimethylaminoacrolein (DMAA) following the method of Jutz 1975 were unsuccessful.

Surprisingly, the Applicant's found that reacting 3-dimethylaminoacrolein (DMAA, III-1) and ABA methyl ester (II-1) in presence of acetic acid and piperidine directly produced the tetralone ABA methyl ester I-1 in a one step reaction.

Accordingly, in an exemplary embodiment, the Applicants have developed a one step process for preparing an ester of (+)-ABA tetralone by reacting an ester of (+)-ABA with dimethylamino acrolein (III-1) in the presence of a suitable organic acid such as acetic acid or propionic acid, a suitable organic amine such as piperidine or morpholine in a suitable inert solvent such as toluene. The Applicants have further shown that products of the process of the application can be further reacted to provide compounds of interest. In some embodiments, the ester of (+)-ABA tetralone is further hydrolyzed to provide (+)-ABA tetralone (+)-5. The Applicants have also shown that the process of the application is reproducible and efficient when scaled up to a gram basis. Accordingly, the process of the application allows for the chemical synthesis of enantiopure products including natural product analogs such as (+)-ABA tetralone (+)-5 in large quantities.

Accordingly, the present application comprises a process for preparing a compound of Formula (I):

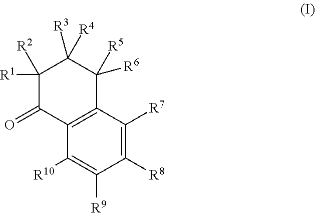

comprising reacting a compound of Formula (II)

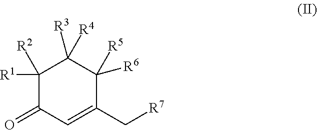

with a compound of Formula (III),

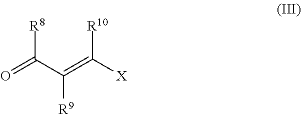

in the presence of a suitable organic acid, suitable organic amine base and a suitable inert solvent, with heating under conditions to remove water,
wherein
R¹, R², R³, R⁴, R⁵ and R⁶ are independently selected from H, halo, OH, CN, C₁₋₃alkyl, C₂₋₄alkenyl, C₂₋₄alkynyl, C₃₋₆cycloalkyl, C₆₋₁₀aryl and C₁₋₃alkyl substituted with one or more OH; wherein each cycloalkyl and aryl optionally substituted with one or more substituents independently selected from OH, halo, C₁₋₆alkyl and OC₁₋₆alkyl;
one of R¹, R², R³, R⁴, R⁵ and R⁶ is further optionally selected from C₃₋₁₀alkyl, C₄₋₁₀alkenyl, C₄₋₁₀alkynyl, ZC₁₋₁₀alkylene, ZC₂₋₄alkenylene, ZC₂₋₄alkynylene, C₁₋₁₀alkyleneZ′, C₂₋₁₀ alkenyleneZ′, C₂₋₄alkynyleneZ′, wherein each alkyl, alkenyl, alkynyl, alkylene, alkenylene and alkynylene is optionally substituted with one or more substituents independently selected from OH, halo, C₁₋₆alkyl and OC₁₋₆alkyl;
each Z is independently selected from O, S, C(O), S(O), SO₂, NR¹¹, NR¹¹C(O) and C(O)NR¹¹;
each Z′ is independently selected from OR¹², CO₂R¹³, C(O)R¹², NR¹²R¹⁴, and C(O)NR¹²R¹⁴;
X is selected from halo, OR¹⁵ and NR¹⁵R¹⁶,
R⁷ is selected from H, halo, OR¹⁷, C(O)R¹⁷, C₁₋₁₀alkyl and C₁₋₁₀alkyl substituted with one or more OH;
R⁸, R⁹ and R¹⁰ are independently selected from H, C₁₋₁₀alkyl, OC₁₋₁₀alkyl, C₆₋₁₀aryl, and C₆₋₁₀aryl optionally substituted with one or more substituents independently selected from OH, halo, C₁₋₆alkyl and OC₁₋₆alkyl;
R¹, R¹², R¹⁴, R¹⁵, R¹⁶ and R¹⁷ are independently selected from H and C₁₋₁₀alkyl; and
R¹³ is C₁₋₁₀alkyl,
provided at least two of R¹, R², R³ and R⁴ are other than H.

In some embodiments, R¹, R², R³, R⁴, R⁵ and R⁶ are independently selected from H, halo, OH, CN, C₁₋₃alkyl, C₁₋₃alkyl substituted with one or more of OH, C₂₋₄alkenyl, C₂₋₄alkynyl, cyclopropyl, cyclobutyl, cyclopentyl, cyclohexyl, and phenyl, the later 5 groups being optionally substituted with one or more of OH, halo, C₁₋₄alkyl and OC₁₋₄alkyl. In some embodiments, R¹, R², R³, R⁴, R⁵ and R⁶ are independently selected from H, halo, OH, CN, C₁₋₃alkyl, C₁₋₃alkyl substituted with one or two of OH, C₂₋₄alkenyl, C₂₋₄alkynyl, cyclopropyl, cyclobutyl, cyclopentyl, cyclohexyl, and phenyl, the later 5 groups being optionally substituted with one or more of OH, halo, C₁₋₄alkyl and OC₁₋₄alkyl. In some embodiments,
R¹, R², R³, R⁴, R⁵ and R⁶ are independently selected from H, halo, OH, CN, C₁₋₃alkyl, C₁₋₃alkyl substituted with one or two of OH, C₂₋₄alkenyl, C₂₋₄alkynyl, cyclopropyl, cyclobutyl, and phenyl, the later 3 groups being optionally substituted with one or more of OH, halo, C₁₋₄alkyl and OC₁₋₄alkyl. In some embodiments, R¹, R², R³, R⁴, R⁵ and R⁶ are independently selected from H, halo, OH, CN, C₁₋₃alkyl, C₁₋₃alkyl substituted with one or two of OH, C₂₋₄alkenyl, C₂₋₄alkynyl, cyclopropyl, phenyl and phenyl substituted with one or more of OH, halo, C₁₋₄alkyl and OC₁₋₄alkyl.

In some embodiments, R¹ and R² are independently selected from H, F, Cl, OH, CN, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R¹ and R² are independently selected from H, OH, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R¹ and R² are independently selected from H, OH, C₁₋₃alkyl, C₁₋₃alkyl substituted with one or two of OH, C₂₋₄alkynyl, and cyclopropyl. In some embodiments, R¹ and R² are independently selected from H, C₁₋₃alkyl, C₂₋₄alkynyl and cyclopropyl. In some embodiments, R¹ and R² are both H. In some embodiments, R¹ and R² are independently selected from C₁₋₃alkyl, C₂₋₄alkynyl and cyclopropyl.

In some embodiments, R³ and R⁴ are independently selected from H, F, Cl, OH, CN, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R³ and R⁴ are independently selected from H, OH, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R³ and R⁴ are independently selected from H, OH, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R³ and R⁴ are independently selected from H, C₁₋₃alkyl, C₁₋₃alkyl substituted with one or two of OH, C₂₋₄alkynyl, cyclopropyl and phenyl optionally substituted with one or more of OH, C₁₋₄alkyl and OC₁₋₄alkyl.

In some embodiments, R³ and R⁴ are independently selected from H, C₁₋₃alkyl, C₁₋₃alkyl substituted with one or two of OH, C₂₋₄alkynyl, cyclopropyl and phenyl optionally substituted with one or two of OH, C₁₋₄alkyl and OC₁₋₄alkyl. In some embodiments, R³ and R⁴ are independently selected from H, C₁₋₃alkyl, C₁₋₃alkyl substituted with one or two of OH, C₂₋₄alkynyl and cyclopropyl. In some embodiments, one of R³ and R⁴ is selected from C₁₋₃alkyl, C₁₋₃alkyl substituted with one or two of OH, C₂₋₄alkynyl and cyclopropyl and the other is selected from H and C₁₋₃alkyl. In some embodiments, R³ and R⁴ are independently selected from C₁₋₃alkyl.

In some embodiments, R¹ and R² are both H and R³ and R⁴ are independently selected from C₁₋₃alkyl, C₁₋₃alkyl substituted with one or two of OH, C₂₋₄alkynyl and cyclopropyl. In some embodiments, R¹ and R² are both H and R³ and R⁴ are independently selected from C₁₋₃alkyl.

In some embodiments, R¹ and R² are selected from C₁₋₃alkyl, C₂₋₄alkynyl and cyclopropyl and R³ and R⁴ are independently selected from H, C₁₋₃alkyl, C₂₋₄alkenyl, C₂₋₄alkynyl, cyclopropyl and phenyl optionally substituted with one or more of OH, C₁₋₄alkyl and OC₁₋₄alkyl.

In some embodiments, R⁵ and R⁶ are independently selected from H, F, Cl, OH, CN, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R⁵ and R⁶ are independently selected from H, OH, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R⁵ and R⁶ are independently selected from H, OH, C₁₋₃alkyl, C₁₋₃alkyl substituted with one or two of OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, R⁵ and R⁶ are independently selected from H, OH, C₁₋₃alkyl, C₂₋₄alkenyl, C₂₋₄alkynyl, cyclopropyl and phenyl optionally substituted with one or more of OH, C₁₋₄alkyl and OC₁₋₄alkyl. In some embodiments, one of R⁵ and R⁶ is selected from H, OH, and C₁₋₃alkyl and the other is selected from H, OH, C₁₋₃alkyl, C₂₋₄alkenyl, C₂₋₄alkynyl, cyclopropyl and phenyl optionally substituted with one or more of OH, C₁₋₄alkyl and OC₁₋₄alkyl.

In some embodiments, one of R¹, R², R³, R⁴, R⁵ and R⁶ is further optionally selected from C₃₋₈alkyl, C₄₋₈alkenyl, C₄₋₈alkynyl, ZC₁₋₈alkylene, ZC₂₋₈alkenylene, ZC₂₋₈alkynylene, C₁₋₈alkyleneZ′, C₂₋₈alkenyleneZ′, C₂₋₈alkynyleneZ′, wherein each alkyl, alkenyl, alkynyl, alkylene, alkenylene and alkynylene is optionally substituted with one or more of OH, halo, C₁₋₆alkyl and OC₁₋₆alkyl.

In some embodiments, one of R¹, R², R³, R⁴, R⁵ and R⁶ is further optionally selected from C₃₋₈alkyl, C₄₋₈alkenyl and C₄₋₈alkynyl, wherein each alkyl, alkenyl and alkynyl is optionally substituted with one or more of OH, halo, C₁₋₆alkyl and OC₁₋₆alkyl. In some embodiments, one of R¹, R², R³, R⁴, R⁵ and R⁶ is further optionally selected from C₃₋₈alkyl, C₄₋₈alkenyl and C₄₋₈alkynyl, wherein each alkyl, alkenyl and alkynyl is optionally substituted with one or more of OH, F, Cl, C₁₋₆alkyl and OC₁₋₆alkyl. In some embodiments, one of R¹, R², R³, R⁴, R⁵ and R⁶ is further optionally selected from C₃₋₈alkyl. In some embodiments, one of R¹, R², R³, R⁴, R⁵ and R⁶ is further optionally selected from C₄₋₈alkenyl and C₄₋₈ alkynyl, wherein each alkenyl and alkynyl is optionally substituted with one or more of OH, F, Cl, C₁₋₆alkyl and OC₁₋₆alkyl. In some embodiments, one of R¹, R², R³, R⁴, R⁵ and R⁶ is further optionally selected from C₄₋₆alkenyl and C₄₋₆alkynyl, wherein each alkenyl and alkynyl is optionally substituted with one or more of OH, C₁₋₆alkyl and OC₁₋₆alkyl. In some embodiments, one of R¹, R², R³, R⁴, R⁵ and R⁶ is further optionally selected from C₄₋₆alkenyl optionally substituted with one or more of OH, C₁₋₆alkyl and OC₁₋₆alkyl. In some embodiments, one of R³, R⁴, R⁵ and R⁶ is further optionally selected from C₄₋₆alkenyl optionally substituted with one or more of OH, C₁₋₆alkyl and OC₁₋₆alkyl. In some embodiments, one of R⁵ and R⁶ is further optionally selected from C₄₋₆alkenyl optionally substituted with one or more of OH, C₁₋₆alkyl and OC₁₋₆alkyl.

In some embodiments, one of R¹, R², R³, R⁴, R⁵ and R⁶ is further optionally selected from ZC₁₋₆alkylene, ZC₂₋₈alkenylene and ZC₂₋₈alkynylene, wherein each alkylene, alkenylene and alkynylene is optionally substituted with one or more substituents independently selected from OH, halo, C₁₋₆alkyl and OC₁₋₆alkyl. In some embodiments, one of R¹, R², R³, R⁴, R⁵ and R⁶ is further optionally selected from ZC₁₋₆alkylene, ZC₂₋₆alkenylene and ZC₂₋₆alkynylene, wherein each alkylene, alkenylene and alkynylene is optionally substituted with one or more substituents independently selected from OH, F, Cl, C₁₋₄alkyl and OC₁₋₄alkyl. In some embodiments, one of R³, R⁴, R⁵ and R⁶ is further optionally selected from ZC₁₋₆alkylene, ZC₂₋₆alkenylene and ZC₂₋₆alkynylene, wherein each alkylene, alkenylene and alkynylene is optionally substituted with one or more substituents independently selected from of OH, F, Cl, C₁₋₄alkyl and OC₁₋₄alkyl. In some embodiments, one of R⁵ and R⁶ is further optionally selected from ZC₁₋₆alkylene, ZC₂₋₆alkenylene and ZC₂₋₆alkynylene, wherein each alkylene, alkenylene and alkynylene is optionally substituted with one or more substituents independently selected from OH, F, Cl, C₁₋₄alkyl and OC₁₋₄alkyl.

In some embodiments, Z is selected from O, S, C(O), S(O) and SO₂. In some embodiments, Z is O. In some embodiments, Z is selected from S, S(O) and SO₂. In some embodiments, Z is selected NR¹¹, NR¹¹C(O) and C(O)NR¹¹. In some embodiments, Z is NR¹¹.

In some embodiments, R¹¹ is selected from H and C₁₋₆alkyl. In some embodiments, R¹¹ is selected from H and C₁₋₄alkyl. In some embodiments, R¹¹ is selected from H, CH₃, CH₂CH₃, CH₂CH₂CH₃, CH(CH₃)₂, CH(CH₃)CH₂CH₃, and CH(CH₃)₃. In some embodiments, R¹¹ is selected from H, CH₃, CH₂CH₃, and CH(CH₃)₂.

In some embodiments, one of R¹, R², R³, R⁴, R⁵ and R⁶ is further optionally selected from C₁₋₆alkyleneZ′, C₂₋₈alkenyleneZ′ and C₂₋₈alkynyleneZ′, wherein each alkylene, alkenylene and alkynylene is optionally substituted with one or more of OH, halo, C₁₋₆alkyl and OC₁₋₆alkyl. In some embodiments, one of R¹, R², R³, R⁴, R⁵ and R⁶ is further optionally selected from C₁₋₆alkyleneZ′, C₂₋₆alkenyleneZ′ and C₂₋₆alkynyleneZ′, wherein each alkylene, alkenylene and alkynylene is optionally substituted with one or more of OH, F, Cl, C₁₋₆alkyl and OC₁₋₆alkyl. In some embodiments, one of R¹, R², R³, R⁴, R⁵ and R⁶ is further optionally selected from C₁₋₆alkyleneZ′, C₂₋₆alkenyleneZ′ and C₂₋₆alkynyleneZ′, wherein each alkylene, alkenylene and alkynylene is optionally substituted with one or more of OH, C₁₋₆alkyl and OC₁₋₆alkyl. In some embodiments, one of R¹, R², R³, R⁴, R⁵ and R⁶ is further optionally selected from C₁₋₆alkyleneZ′, C₂₋₆alkenyleneZ′ and C₂₋₆alkynyleneZ′, wherein each alkylene, alkenylene and alkynylene is optionally substituted with one to three of OH, C₁₋₆alkyl and OC₁₋₆alkyl. In some embodiments, one of R¹, R², R³, R⁴, R⁵ and R⁶ is further optionally selected from C₂₋₆alkenyleneZ′ and C₂₋₆alkynyleneZ′, wherein each alkenylene and alkynylene is optionally substituted with one to three of OH, C₁₋₆alkyl and OC₁₋₆alkyl.

In some embodiments, one of R³, R⁴, R⁵ and R⁶ is further optionally selected from C₂₋₆alkenyleneZ′ and C₂₋₆alkynyleneZ′, wherein each alkenylene and alkynylene is optionally substituted with one to three substituents selected from OH, C₁₋₆alkyl and OC₁₋₆alkyl. In some embodiments, one of R⁵ and R⁶ is further optionally selected from C₂₋₆alkenyleneZ′ and C₂₋₆alkynyleneZ′, wherein each alkenylene and alkynylene is optionally substituted with one to three substituents selected from OH, C₁₋₆alkyl and OC₁₋₆alkyl. In some embodiments, one of R⁵ and R⁶ is further optionally selected from C₂₋₆alkenyleneZ′ optionally substituted with one to three substituents selected from OH, C₁₋₆alkyl and OC₁₋₆alkyl. In some embodiments, one of R⁵ and R⁶ is further optionally selected from C₂₋₆alkenyleneZ′ optionally substituted with one or two of C₁₋₄alkyl. In some embodiments, R⁶ is C₂₋₆alkenyleneZ′ optionally substituted with one or two of C₁₋₄alkyl.

In some embodiments, Z′ is NR¹²R¹⁴. In some embodiments, Z′ is C(O)NR¹²R¹⁴. In some embodiments, Z′ is selected from OR¹², CO₂R¹³ and C(O)R¹². In some embodiments, Z′ is selected from OR¹² and C(O)R¹². In some embodiments, Z′ is CO₂R¹³.

In some embodiments, one of R¹, R², R³, R⁴, R⁵ and R⁶ is further optionally selected from C₁₋₆alkyleneZ′, C₂₋₆alkenyleneZ′ and C₂₋₆alkynyleneZ′, wherein each alkylene, alkenylene and alkynylene is optionally substituted with one to three substituents selected from OH, C₁₋₆alkyl and OC₁₋₆alkyl and Z′ is CO₂R¹³. In some embodiments, one of R³, R⁴, R⁵ and R⁶ is further optionally selected from C₁₋₆alkyleneZ′, C₂₋₆alkenyleneZ′ and C₂₋₆alkynyleneZ′, wherein each alkylene, alkenylene and alkynylene is optionally substituted with one to three substituents selected from OH, C₁₋₆alkyl and OC₁₋₆alkyl and Z′ is CO₂R¹³. In some embodiments, one of R⁵ and R⁶ is further optionally selected from C₁₋₆alkyleneZ′, C₂₋₆alkenyleneZ′ and C₂₋₆alkynyleneZ′, wherein each alkylene, alkenylene and alkynylene is optionally substituted with one to three substituents selected from OH, C₁₋₆alkyl and OC₁₋₆alkyl and Z′ is CO₂R¹³.

In some embodiments, one of R⁵ and R⁶ is selected from H, OH, and C₁₋₃alkyl and the other is selected from C₁₋₆alkyleneZ′, C₂₋₆alkenyleneZ′ and C₂₋₆alkynyleneZ′, wherein each alkylene, alkenylene and alkynylene is optionally substituted with one to three of OH, C₁₋₆alkyl and OC₁₋₆alkyl and Z′ is CO₂R¹³. In some embodiments, one of R⁵ and R⁶ is selected from H, OH, and C₁₋₃alkyl and the other is selected from C₂₋₆alkenyleneZ′ and C₂₋₆alkynyleneZ′, wherein each alkenylene and alkynylene is optionally substituted with one to three of OH, C₁₋₆alkyl and OC₁₋₆alkyl and Z′ is CO₂R¹³. In some embodiments, one of R⁵ and R⁶ is selected from OH, and C₁₋₃alkyl and the other is selected from C₂₋₆alkenyleneZ′ optionally substituted with one to three of OH, C₁₋₆alkyl and OC₁₋₆alkyl and Z′ is CO₂R¹³. In some embodiments, one of R⁵ and R⁶ is OH and the other is

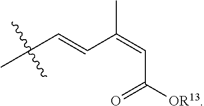

In some embodiments, R¹² and R¹⁴ are independently selected from H and C₁₋₆alkyl. In some embodiments, R¹² and R¹⁴ are independently selected from H and C₁₋₄alkyl. In some embodiments, R¹² and R¹⁴ are independently selected from H, CH₃, CH₂CH₃, CH₂CH₂CH₃, CH(CH₃)₂, CH(CH₃)CH₂CH₃, and CH(CH₃)₃. In some embodiments,
R¹² and R¹⁴ are independently selected from H, CH₃, CH₂CH₃, and CH(CH₃)₂.

In some embodiments, R¹³ is C₁₋₆alkyl. In some embodiments, R¹³ is C₁₋₄alkyl. In some embodiments, R¹³ is selected from CH₃, CH₂CH₃, CH₂CH₂CH₃, CH(CH₃)₂, CH(CH₃)CH₂CH₃, and CH(CH₃)₃. In some embodiments, R¹³ is selected from H, CH₃, CH₂CH₃, and CH(CH₃)₂.

In some embodiments, X is selected from Cl, Br, I, OR¹⁵ and NR¹⁵R¹⁶. In some embodiments, X is selected from Cl, Br and I. In some embodiments, X is OR¹⁵. In some embodiments, X is NR¹⁵R¹⁶.

In some embodiments, R¹⁵ and R¹⁶ are independently selected from H and C₁₋₆alkyl. In some embodiments, R¹⁵ and R¹⁶ are independently selected from H and C₁₋₄alkyl. In some embodiments, R¹⁵ and R¹⁶ are independently selected from H, CH₃, CH₂CH₃, CH₂CH₂CH₃, CH(CH₃)₂, CH(CH₃)CH₂CH₃, and CH(CH₃)₃. In some embodiments, R¹⁵ and R¹⁶ are independently selected from H, CH₃, CH₂CH₃, and CH(CH₃)₂. In some embodiments, R¹⁵ and R¹⁶ are independently selected from CH₃, CH₂CH₃, and CH(CH₃)₂. In some embodiments, R¹⁵ and R¹⁶ are both CH₃.

In some embodiments, R⁷ is selected from H, halo, OR¹⁷, C(O)R¹⁷, C₁₋₈alkyl and C₁₋₈alkyl substituted with one or more OH. In some embodiments, R⁷ is selected from H, F, Br, Cl, OR¹⁷, C(O)R¹⁷, C₁₋₈alkyl and C₁₋₈alkyl substituted with one or more OH. In some embodiments, R⁷ is selected from F, Br and Cl. In some embodiments, R⁷ is selected from H, C₁₋₈alkyl and C₁₋₈alkyl substituted with one or two OH. In some embodiments, R⁷ is H. In some embodiments, R⁷ is C₁₋₈alkyl and C₁₋₈alkyl substituted with one or two OH. In some embodiments, R⁷ is selected from OR¹⁷ and C(O)R¹⁷.

In some embodiments, R¹⁷ is selected from H and C₁₋₆alkyl. In some embodiments, R¹⁷ is selected from H and C₁₋₄alkyl. In some embodiments, R¹⁷ is selected from H, CH₃, CH₂CH₃, CH₂CH₂CH₃, CH(CH₃)₂, CH(CH₃)CH₂CH₃, and CH(CH₃)₃. In some embodiments, R¹⁷ is selected from H, CH₃, CH₂CH₃, and CH(CH₃)₂. In some embodiments, R¹⁷ is H.

In some embodiments, R⁸, R⁹ and R¹⁰ are independently selected from H, C₁₋₈alkyl, OC₁₋₈alkyl, phenyl, and phenyl substituted with one or more substituents independently selected from OH, halo, C₁₋₄alkyl and OC₁₋₄alkyl. In some embodiments, R⁸,
R⁹ and R¹⁰ are independently selected from H, C₁₋₆alkyl, OC₁₋₆alkyl, phenyl, and phenyl optionally substituted with one or more substituents independently selected from OH, halo, C₁₋₄alkyl and OC₁₋₄alkyl. In some embodiments, R⁸, R⁹ and R¹⁰ are independently selected from H, C₁₋₆alkyl, and OC₁₋₆alkyl.

In some embodiments, R¹ and R² are both H and the present application includes a process of preparing a compound of Formula (I-A):

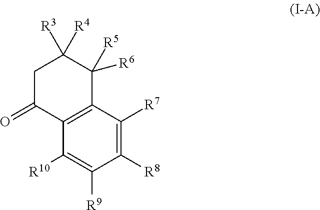

comprising reacting a compound of Formula (II)

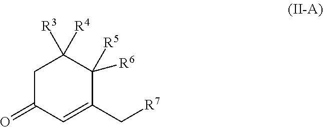

with a compound of Formula (III),

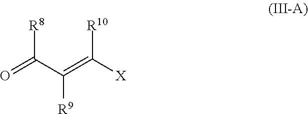

in the presence of a suitable organic acid, suitable organic amine base and a suitable inert solvent,
with heating under conditions to remove water,
wherein:
   R³ and R⁴ are independently selected from halo, OH, CN, C₁₋₃alkyl, C₂₋₄alkenyl, C₂₋₄alkynyl, C₃₋₆cycloalkyl, C₆₋₁₀aryl, C₁₋₃alkyl substituted with OH; wherein each cycloalkyl and aryl optionally substituted with one or more substituents independently selected from of OH, halo, C₁₋₆alkyl and OC₁₋₆alkyl;
   R⁵ is selected from H, halo, OH, CN, C₁₋₃alkyl, C₂₋₄alkenyl, C₂₋₄alkynyl, C₃₋₆cycloalkyl, C₆₋₁₀aryl, and C₁₋₃alkyl substituted with OH; wherein each cycloalkyl and aryl optionally substituted with one or more substituents independently selected from OH, halo, C₁₋₆alkyl and OC₁₋₆alkyl;
   R⁶ is selected from C₃₋₁₀alkyl, C₄₋₁₀alkenyl, C₄₋₁₀alkynyl, ZC₁₋₁₀alkylene, ZC₂₋₁₀ alkenylene, ZC₂₋₄alkynylene, C₁₋₁₀alkyleneZ′, C₂₋₄alkenyleneZ′, C₂₋₄alkynyleneZ′, wherein each alkyl, alkenyl, alkynyl, alkylene, alkenylene and alkynylene is optionally substituted with one or more substituents independently selected from OH, halo, C₁₋₆alkyl and OC₁₋₆alkyl; and
   Z is independently selected from O, S, C(O), S(O), SO₂, NR¹¹, NR¹¹C(O) and C(O)NR¹¹;
   Z′ is independently selected from OR¹², CO₂R¹³, C(O)R¹², NR¹²R¹⁴, and C(O)NR¹²R¹⁴;
   X is selected from halo, OR¹⁵ and NR¹⁵R¹⁶;
   R⁷ is selected from H, halo, OR¹⁷, C(O)R¹⁷, C₁₋₁₀alkyl and C₁₋₁₀alkyl substituted with one or more OH;
   R⁸, R⁹ and R¹⁰ are independently selected from H, C₁₋₁₀alkyl, OC₁₋₁₀alkyl, C₆₋₁₀aryl, and C₆₋₁₀aryl optionally substituted with one or more substituents independently selected from OH, halo, C₁₋₆alkyl and OC₁₋₆alkyl;
   R¹¹, R¹², R¹⁴, R¹⁵, R¹⁶ and R¹⁷ are independently selected from H and C₁₋₁₀alkyl; and R¹³ is C₁₋₁₀alkyl.

In some embodiments, R³ and R⁴ are independently selected from F, Cl, OH, CN, C₁₋₃alkyl. C₁₋₃alkyl substituted with OH, C₂₋₄alkenyl, C₂₋₄alkynyl, cyclopropyl and phenyl optionally substituted with one or more of OH, halo, C₁₋₄alkyl and OC₁₋₄alkyl. In some embodiments, one of R³ and R⁴ is selected from C₁₋₃alkyl, C₁₋₃alkyl substituted with OH, C₂₋₄alkynyl and cyclopropyl and the other is C₁₋₃alkyl. In some embodiments, R³ and R⁴ are independently selected from C₁₋₃alkyl.

In some embodiments, R⁵ is independently selected from H, OH, C₁₋₃alkyl, C₂₋₄alkenyl, C₂₋₄alkynyl, cyclopropyl and phenyl optionally substituted with one or more of OH, C₁₋₄alkyl and OC₁₋₄alkyl. In some embodiments, R⁵ is selected from H, OH, and C₁₋₃alkyl.

In some embodiments, R⁶ is selected from C₃₋₈alkyl, C₄₋₈alkenyl, C₄₋₈alkynyl, ZC₁₋₆alkylene, ZC₂₋₈alkenylene, ZC₂₋₈alkynylene, C₁₋₆alkyleneZ′, C₂₋₈alkenyleneZ′, C₂₋₈ alkynyleneZ′, wherein each alkyl, alkenyl, alkynyl, alkylene, alkenylene and alkynylene is optionally substituted with one or more substituents independently selected from OH, halo, C₁₋₆alkyl and OC₁₋₆alkyl.

In some embodiments, R⁶ is selected from C₃₋₈alkyl, C₄₋₈alkenyl and C₄₋₈ alkynyl, wherein each alkyl, alkenyl and alkynyl is optionally substituted with one or more substituents independently selected from OH, halo, C₁₋₆alkyl and OC₁₋₆alkyl. In some embodiments, R⁶ is selected from C₄₋₆alkenyl and C₄₋₆alkynyl, wherein each alkenyl and alkynyl is optionally substituted with one or more substituents independently selected from OH, C₁₋₆alkyl and OC₁₋₆alkyl. In some embodiments, R⁶ is C₄₋₆alkenyl optionally substituted with one or more substituents independently selected from OH, C₁₋₆alkyl and OC₁₋₆alkyl.

In some embodiments, R⁶ is selected from ZC₁₋₆alkylene, ZC₂₋₈alkenylene and ZC₂₋₈alkynylene, wherein each alkylene, alkenylene and alkynylene is optionally substituted with one or more substituents independently selected from OH, halo, C₁₋₆alkyl and OC₁₋₆alkyl. In some embodiments, R⁶ is selected from ZC₁₋₆alkylene, ZC₂₋₆alkenylene and ZC₂₋₆alkynylene, wherein each alkylene, alkenylene and alkynylene is optionally substituted with one or more substituents independently selected from OH, F, Cl, C₁₋₄alkyl and OC₁₋₄alkyl.

In some embodiments, Z is selected from O, S, C(O), S(O) and SO₂. In some embodiments, Z is O. In some embodiments, Z is selected from S, S(O) and SO₂. In some embodiments, Z is selected NR¹¹, NR¹¹C(O) and C(O)NR¹¹. In some embodiments, Z is NR¹¹.

In some embodiments, R¹¹ is selected from H and C₁₋₆alkyl. In some embodiments, R¹¹ is selected from H and C₁₋₄alkyl. In some embodiments, R¹¹ is selected from H, CH₃, CH₂CH₃, CH₂CH₂CH₃, CH(CH₃)₂, CH(CH₃)CH₂CH₃, and CH(CH₃)₃. In some embodiments, R¹¹ is selected from H, CH₃, CH₂CH₃, and CH(CH₃)₂.

In some embodiments, R⁶ is selected from C₁₋₈alkyleneZ′, C₂₋₈alkenyleneZ′ and C₂₋₈alkynyleneZ′, wherein each alkylene, alkenylene and alkynylene is optionally substituted with one or more of OH, halo, C₁₋₆alkyl and OC₁₋₆alkyl. In some embodiments, R⁶ is selected from C₁₋₆alkyleneZ′, C₂₋₆alkenyleneZ′ and C₂₋₆alkynyleneZ′, wherein each alkylene, alkenylene and alkynylene is optionally substituted with one to three of OH, C₁₋₆ alkyl and OC₁₋₆alkyl. In some embodiments, R⁶ is selected from C₂₋₆alkenyleneZ′ and C₂₋₆alkynyleneZ′, wherein each alkenylene and alkynylene is optionally substituted with one to three of OH, C₁₋₆alkyl and OC₁₋₆alkyl. In some embodiments, R⁶ is C₂₋₆alkenyleneZ′ optionally substituted with one to three of OH, C₁₋₆alkyl and OC₁₋₆alkyl. In some embodiments, R⁶ is C₂₋₆alkenyleneZ′ optionally substituted with one or two of C₁₋₄alkyl.

In some embodiments, Z′ is NR¹²R¹⁴. In some embodiments, Z′ is C(O)NR¹²R¹⁴. In some embodiments, Z′ is selected from OR¹², CO₂R¹³ and C(O)R¹². In some embodiments, Z′ is selected from OR¹² and C(O)R¹². In some embodiments, Z′ is CO₂R¹³.

In some embodiments, R⁶ is selected from C₁₋₆alkyleneZ′, C₂₋₆alkenyleneZ′ and C₂₋₆alkynyleneZ′, wherein each alkylene, alkenylene and alkynylene is optionally substituted with one to three of OH, C₁₋₆alkyl and OC₁₋₆alkyl and Z′ is CO₂R¹³.

In some embodiments, R⁶ is C₂₋₆alkenyleneZ′ optionally substituted with one to three of OH, C₁₋₆alkyl and OC₁₋₆alkyl and Z′ is CO₂R¹³. In some embodiments, R⁶ is

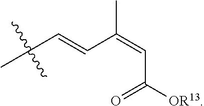

In some embodiments, R¹² and R¹⁴ are independently selected from H and C₁₋₆alkyl. In some embodiments, R¹² and R¹⁴ are independently selected from H and C₁₋₄alkyl. In some embodiments, R¹² and R¹⁴ are independently selected from H, CH₃, CH₂CH₃, CH₂CH₂CH₃, CH(CH₃)₂, CH(CH₃)CH₂CH₃, and CH(CH₃)₃. In some embodiments, R¹² and R¹⁴ are independently selected from H, CH₃, CH₂CH₃, and CH(CH₃)₂.

In some embodiments, R¹³ is C₁₋₆alkyl. In some embodiments, R¹³ is C₁₋₄alkyl. In some embodiments, R¹³ is selected from CH₃, CH₂CH₃, CH₂CH₂CH₃, CH(CH₃)₂, CH(CH₃)CH₂CH₃, and CH(CH₃)₃. In some embodiments, R¹³ is selected from H, CH₃, CH₂CH₃, and CH(CH₃)₂.

In some embodiments, R⁵ is selected from H, OH, and C₁₋₃alkyl and R⁶ is C₂₋₆alkenyleneZ′ optionally substituted with one to three of OH, C₁₋₆alkyl and OC₁₋₆alkyl and Z′ is CO₂R¹³.

In some embodiments, the compound of Formula II is selected from the compounds listed below:

| Compound I.D | Structure |
|---|---|
| II-1 | 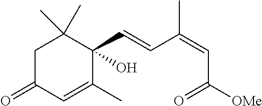 [SMILES: COC(=O)/C=C(C)\C=C\[C@@]1(O)C(C)=CC(=O)CC1(C)C] |
| II-2 | 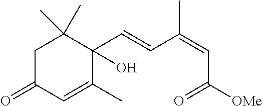 [SMILES: COC(=O)/C=C(C)\C=C\C1(O)C(C)=CC(=O)CC1(C)C] |
| II-3 | 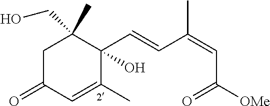 [SMILES: COC(=O)/C=C(C)\C=C\[C@@]1(O)C(C)=CC(=O)C[C@]1(C)CO] |
| II-4 | 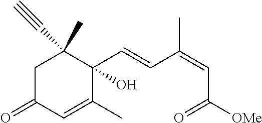 [SMILES: C#C[C@@]1(C)CC(=O)C=C(C)[C@]1(O)/C=C/C(C)=C\C(=O)OC] |

In some embodiments, X is selected from Cl, Br, I, OR¹⁵ and NR¹⁵R¹⁶. In some embodiments, X is selected from Cl, Br and I. In some embodiments, X is OR¹⁵. In some embodiments, X is NR¹⁵R¹⁶.

In some embodiments, R¹⁵ and R¹⁶ are independently selected from H and C₁₋₆alkyl. In some embodiments, R¹⁵ and R¹⁶ are independently selected from H and C₁₋₄alkyl. In some embodiments, R¹⁵ and R¹⁶ are independently selected from H, CH₃, CH₂CH₃, CH₂CH₂CH₃, CH(CH₃)₂, CH(CH₃)CH₂CH₃, and CH(CH₃)₃. In some embodiments, R¹⁵ and R¹⁶ are independently selected from H, CH₃, CH₂CH₃, and CH(CH₃)₂. In some embodiments, R¹⁵ and R¹⁶ are independently selected from CH₃, CH₂CH₃, and CH(CH₃)₂. In some embodiments, R¹⁵ and R¹⁶ are both CH₃.

In some embodiments, R⁷ is selected from H, halo, OR¹⁷, C(O)R¹⁷, C₁₋₈alkyl and C₁₋₈alkyl substituted with one or more OH. In some embodiments, R⁷ is selected from H, F, Br, Cl, OR¹⁷, C(O)R¹⁷, C₁₋₈alkyl and C₁₋₈alkyl substituted with one or more OH. In some embodiments, R⁷ is selected from F, Br and Cl. In some embodiments, R⁷ is selected from H, C₁₋₈alkyl and C₁₋₈alkyl substituted with one or two OH. In some embodiments, R⁷ is H. In some embodiments, R⁷ is C₁₋₈alkyl and C₁₋₈alkyl substituted with one or two OH. In some embodiments, R⁷ is selected from OR¹⁷ and C(O)R¹⁷.

In some embodiments, R¹⁷ is selected from H and C₁₋₆alkyl. In some embodiments, R¹⁷ is selected from H and C₁₋₄alkyl. In some embodiments, R¹⁷ is selected from H, CH₃, CH₂CH₃, CH₂CH₂CH₃, CH(CH₃)₂, CH(CH₃)CH₂CH₃, and CH(CH₃)₃. In some embodiments, R¹⁷ is selected from H, CH₃, CH₂CH₃, and CH(CH₃)₂. In some embodiments, R¹⁷ is H.

In some embodiments, R⁸, R⁹ and R¹⁰ are independently selected from H, C₁₋₈alkyl, OC₁₋₈alkyl, phenyl, and phenyl optionally substituted with one or more of OH, halo, C₁₋₄alkyl and OC₁₋₄alkyl. In some embodiments, R⁸, R⁹ and R¹⁰ are independently selected from H, C₁₋₆alkyl, OC₁₋₆alkyl, phenyl, and phenyl optionally substituted with one or more of OH, halo, C₁₋₄alkyl and OC₁₋₄alkyl. In some embodiments, R⁸, R⁹ and R¹⁰ are independently selected from H, C₁₋₆alkyl, and OC₁₋₆alkyl.

In some embodiments, the compound of Formula III is selected from the compounds listed below:

| Compound I.D | Structure |
|---|---|
| III-1 | 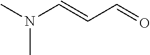 |

In some embodiments, the preparing a compound of Formula I or I-A comprising reacting the compound of Formula II or II-A with an excess amount of a compound of Formula III or III-A, respectively. In some embodiments, the process comprises reacting a compound of Formula II or II-A with about 1.2 to about 3, about 1.2 to about 2.5, about 1.5 to about 2.5, about 1.6 to about 2, or about 2 molar equivalents of a compound of Formula III or III-A, respectively. In some embodiments, the process comprises reacting a compound of Formula II or II-A with about 1.5 to about 2.5, about 1.6 to about 2, or about 1.8 molar equivalents of a compound of Formula III or III-A, respectively.

In some embodiments, the suitable organic acid is any suitable organic carboxylic acid. In some embodiments, the suitable organic acid is a high-boiling point carboxylic acid. In some embodiments, the high-boiling point carboxylic acid has a boiling point of greater than about 70° C., about 80° C., about 90° C., about 100° C., about 110° C., about 115° C., about 125° C., about 135° C., about 150° C. or about 160° C. In some embodiments, the organic acid has a boiling point of about 70° C. to about 210° C., about 80° C. to about 190° C., about 100° C. to about 190° C., about 110° C. to about 165° C. about 110° C. to about 145° C. or about 110° C. to about 145° C. In some embodiments, the organic acid has a boiling point of about 110° C. to about 165° C., about 110° C. to about 145° C. or about 110° C. to about 145° C. In some embodiments, the suitable organic carboxylic acid is selected from acetic acid, formic acid, propionic acid, valeric acid, and butyric acid, and mixtures thereof. In some embodiments, the suitable organic acid is selected from propionic acid and acetic acid and mixtures thereof. In some embodiments, the suitable organic acid comprises propionic acid. In some embodiments, the suitable organic acid comprises acetic acid. In some embodiments, the suitable organic acid is propionic acid. In some embodiments, the suitable organic acid is acetic acid.

In some embodiments, the suitable organic base is any suitable organic amine base. In some embodiments, the suitable organic amine base is high-boiling point organic amine base. In some embodiments, the high-boiling point organic amine base has a boiling point of greater than about 70° C., about 80° C., about 90° C., about 100° C., about 110° C., about 115° C., about 125° C., about 135° C., about 150° C. or about 160° C. In some embodiments, the organic amine base has a boiling point of about 70° C. to about 210° C., about 80° C. to about 190° C., about 100° C. to about 190° C., about 100° C. to about 165° C. about 100° C. to about 145° C. or about 10° C. to about 145° C. In some embodiments, the high-boiling organic amine base has a boiling point of about 100° C. to about 165° C., about 110° C. to about 145° C. or about 100° C. to about 145° C. In some embodiments, the suitable organic amine base is selected from dipropylamine, triethylamine, aminotoluene, aminobenzene, piperidine, methylpiperidine, tetramethylpiperidine, piperazine, methyl piperazine, and morpholine, and mixtures thereof. In some embodiments, the suitable organic amine base is a suitable secondary organic amine base. In some embodiments, the suitable secondary organic amine base selected from piperidine, methylpiperidine, tetramethylpiperidine, piperazine, methyl piperazine, and morpholine, and mixtures thereof. In some embodiments, the suitable secondary organic amine base selected from piperidine, methylpiperidine tetramethylpiperidine, and morpholine, and mixtures thereof. In some embodiments, the suitable secondary organic amine base selected from piperidine and morpholine, and mixtures thereof.

In some embodiments, the process comprises reacting the compound of Formula II or II-A with a compound of Formula III or III-A, respectively, in the presence of about 3 to about 6, about 3 to about 5, or about 4 to about 5 equivalents or about 4 equivalents of the suitable acid relative to the suitable base.

In some embodiments, the ratio (molar equivalents) of the suitable acid to the suitable base is from about 6:1 to about 1:1. In some embodiments, the ratio of the suitable acid to the suitable base is about 5:1, about 4.5:1, about 4:1, about 3:1 or about 2:1. In some embodiments, the ratio of the suitable acid to the suitable base is about 4:1.

In some embodiments, the compounds of Formula II or II-A and Formula III or III-A, respectively are added to a pre-made solution comprising the suitable acid and the suitable base in the suitable inert solvent. In some embodiments, the compound of Formula II is added to the pre-made solution first, followed by the compound of Formula III.

In some embodiments, the conditions to remove water comprise distillation. In some embodiments, the conditions to remove water comprise using a cold finger. In some embodiments, the compound of Formula II or II-A is reacted with the compound of Formula III or III-A, respectively, with heating using a cold finger or a condenser.

In some embodiments, the conditions to remove water comprise azeotropic distillation conditions. In some embodiments, the conditions to remove water comprise azeotropic distillation conditions at about atmospheric pressure. In some embodiments, the conditions to remove water comprise azeotropic distillation conditions at the boiling point of the inert solvent or co-distillation temperature of the azeotrope formed between the inert solvent and water. In some embodiments, the process is driven by the removal of water under the azeotropic distillation conditions. Therefore, in some embodiments, the compound of Formula II or II-A is reacted with the compound of Formula III or III-A with heating under azeotropic distillation conditions. In some embodiments, the conditions to remove water comprise azeotropic distillation conditions using a Dean-Stark trap or any similar apparatus to separate the water formed during the course of the reaction and to return to the reaction site with the suitable inert solvent.

In some embodiments, the conditions to remove water comprise the use of water scavengers. In some embodiments, the water scavenger is an ortho ester compound. In some embodiments, the water scavenger is trimethyl orthoformate. In some embodiments, the water scavenger is not acetic anhydride.

In some embodiments, the suitable inert solvent is a high-boiling-point-solvent. In some embodiments, the high-boiling-point-solvent has a boiling point of greater than about 70° C., about 80° C., about 90° C., about 100° C., about 110° C., about 115° C., about 125° C., about 135° C., about 150° C. or about 160° C. In some embodiments, the high-boiling-point-solvent has a boiling point of about 70° C. to about 170° C., about 80° C. to about 160° C., about 80° C. to about 150° C., about 100° C. to about 150° C. or about 110° C. to about 150° C. In some embodiments, the high-boiling-point-solvent has a boiling point of about 80° C. to about 150° C., about 100° C. to about 150° C. or about 110° C. to about 150° C.

In some embodiments, the suitable inert solvent is any inert organic solvent which azeotropes with water. In some embodiments, the inert solvent azeotropes with water at a temperature below 100° C. In some embodiments, the inert solvent has a boiling point of greater than about 100° C. and azeotropes with water at a temperature below 100° C.

In some embodiments, the suitable inert solvent is selected from heptane cycloalkane, dimethyl carbonate, clorobenzene, benzene, toluene, and xylenes, and mixtures thereof. In some embodiments, the suitable inert solvent is an aromatic solvent. In some embodiments, the aromatic solvent is selected from chlorobenzene, benzene, toluene, and xylenes, and mixtures thereof. In some embodiments, the xylene is m-xylene. In some embodiments, the inert solvent comprises toluene. In some embodiments, the inert solvent is toluene.

In some embodiments, the heating is performed at a temperature of about 75° C. to about 170° C., about 80° C. to about 150° C., about 80° C. to about 140° C., about 135° C., about 125° C. about 84° C.

Therefore, in some embodiments, the step of heating is performed at azeotropic reflux temperature or co-distillation temperature of the azeotrope formed between the inert solvent and water formed during the course of the reaction. In some embodiments, the step of heating is performed at the boiling point of the inert solvent to permit azeotropic removal of the water formed during the course of the reaction.

In some embodiments, the suitable inert solvent is toluene and the azeotropic reflux temperature or co-distillation temperature of the azeotrope formed between toluene and water is about 84° C. Therefore, in some embodiments, the heating is performed at a temperature of about 84° C.

In some embodiments, the compound of Formula II or II-A is reacted with the compound of Formula III or III-A with heating under conditions to remove water for a time to provide the compound of Formula I. In some embodiments, the compound Formula II or II-A is reacted with the compound of Formula III or III-A with heating under conditions to remove water about 6 hours to 36 hours, about 12 to 30 hours, about 16 to 30 hours, about 18 to about 26 hours, about 20 to about 26 hours or about 24 hours to provide the compound of Formula I or I-A.

In some embodiments, the step of reacting the compound of Formula II or II-A with the compound of Formula III or III-A comprises combining the compound of Formula II or II-A and the compound of Formula III or III-A in the presence of an amount of the suitable acid and the suitable base in the suitable inert solvent with heating to form a reaction mixture and adding an additional amount of the compound of Formula III or III-A to the reaction mixture over a time. In some embodiments, additional compound of Formula III or III-A is added in divided amounts over time. In some embodiments, the additional amount of the compound of Formula III or III-A is added in two to four divided amounts over time. In some embodiments, the additional amount of the compound of Formula III or III-A is added in two divided amounts overtime. In some embodiments, the step of reacting the compound of Formula II or II-A with the compound of Formula III or III-A further comprises adding an additional amount of the suitable acid and/or the suitable base to the reaction mixture over a time. In some embodiments, the additional amount of the suitable acid and/or the suitable base is added in two to four divided amounts over time. In some embodiments, the additional amount of the suitable acid and/or the suitable base is added in two divided amounts over time. In some embodiments, the additional amount of the compound of Formula III or III-A and/or the additional amount of the suitable acid and/or the suitable base is added to the reaction mixture over about 8 hours to about 24 hours.

In some embodiments, the compound of Formula II or II-A is combined with about 0.5 to about 1.5 or about 1 molar equivalent of the compound of Formula III or III-A in the presence of an amount of the suitable acid and the suitable base in the suitable inert solvent with heating to form a reaction mixture. In some embodiments, the additional amount of the compound of Formula III or III-A is about 0.75 to about 1.5, or about 0.75 to about 1.25 or about 1 molar equivalent of the compound of Formula III or III-A.

In some embodiments, the process for preparing the compound of Formula I or I-A comprises reacting the compound of Formula II or II-A with the compound of Formula III or Ill-A respectively with heating under conditions to remove water to form a reaction mixture comprising the compound of Formula I or I-A and isolating the compound of Formula I or I-A from the reaction mixture. In some embodiments, the step of isolating the compound of Formula I or I-A from the reaction mixture comprises cooling the reaction mixture, quenching the reaction mixture with a base, extracting the compound of Formula I or I-A from the reaction mixture with an organic solvent to obtain an organic layer comprising the compound of Formula I or I-A and concentrating the organic layer, for example, by evaporation such as roto-evaporation, to provide a crude product comprising the compound of Formula I or I-A.

In some embodiments, the reaction mixture is cooled to a temperature of about 5° C. to about 50° C., about 10° C. to about 30° C. or about 18° C. to about 25° C. (room temperature). In some embodiments, the cooling occurs rapidly or over a specific time period such as about 1 hour to about 24 hours.

In some embodiments, the base for quenching the reaction mixture is an aqueous basic solution. In some embodiments, the aqueous basic solution is a NaHCO₃ solution. In some embodiments, the organic solvent for extracting the compound of Formula I or I-A is ethyl acetate.

In some embodiments, the crude product comprising the compound of Formula I or I-A is purified using chromatography such as column chromatography using a suitable solvent or mixture of solvents, or any other known purification method. In an embodiment, the crude product is purified by fractionation using chromatography such as column chromatography. In some embodiments, the column chromatography is flash column chromatography. In some embodiments, the suitable mixture of solvents for column chromatography is ethyl acetate and hexanes.

In some embodiments, the compound of Formula I is selected from the compounds listed below:

| Compound I.D | Structure |
|---|---|
| I-1 | 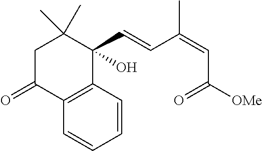 [SMILES: COC(=O)/C=C(C)\C=C\[C@@]1(O)c2ccccc2C(=O)CC1(C)C] |
| I-2 | 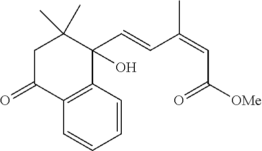 [SMILES: COC(=O)/C=C(C)\C=C\C1(O)c2ccccc2C(=O)CC1(C)C] |
| I-3 | 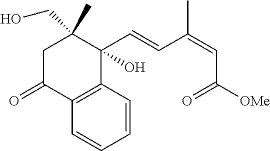 [SMILES: COC(=O)/C=C(C)\C=C\[C@@]1(O)c2ccccc2C(=O)C[C@]1(C)CO] |
| I-4 | 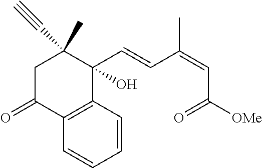 [SMILES: C#C[C@@]1(C)CC(=O)c2ccccc2[C@]1(O)/C=C/C(C)=C\C(=O)OC] |

In some embodiments, the application comprises preparing a compound of Formula I-1 to I-4 comprising reacting a compound of Formula II-1 to II-4, respectively, with a compound of Formula III-1 in presence of a suitable organic acid, suitable organic amine base and a suitable inert solvent, with heating under conditions to remove water.

In some embodiments, the process selectively forms the compound of Formula I or I-A as the major product. Accordingly, in some embodiments, the present application also includes a process for selectively preparing a compound of Formula I or I-A comprising reacting a compound of Formula II or II-A with a compound of Formula III or Ill-A with heating under condition to remove water wherein the compounds of Formulae I, I-A, II, II-A, III and III-A are as defined above.

In some embodiments, the process provides the compound of Formula I or I-A as the as the major product of the process. In some embodiments, the process provides the compound of Formula I or I-A in a yield of greater than about 15%, about 20%, about 25%, about 30%, about 35%, about 40%, about 45%, about 50%, about 55%, about 60%, about 65%, about 70%, about 75%, about 80%, about 85%, about 90% or about 95%. In some embodiments, the process provides the compound of Formula I or I-A in a yield of greater an about 55%, about 60%, about 65%, about 70%, about 75%, about 80%, about 85%, about 90% or about 95%. In some embodiments, the process provides the compound o Formula (I) in a yield of greater an about 70%, about 75%, about 80%, about 85%, about 90% or about 95%. In some embodiments, the process provides the compound of Formula (I) in a yield of greater than about 80%, about 85% or about 90%.

In some embodiments, the compound of Formula I or I-A can be further reacted to form compounds of interest. Therefore, in some embodiments, the compounds of Formula I or I-A are intermediates.

For example, in some embodiments, when one of R¹, R², R³, R⁴, R⁵ and R⁶ is selected from C₁₋₁₀alkyleneZ′, C₂₋₄alkenyleneZ′, C₂₋₄alkynyleneZ and Z′ is CO₂R¹³ such as in the compounds of Formula I-1 to I-4, then the compound of Formula I or I-A is further hydrolyzed, for example, in the presence of a base to provide the free carboxylic acid derivative of the compound of Formula I or I-A. Therefore, when one of R¹, R², R³, R⁴, R⁵ and R⁶ is selected from C₁₋₁₀alkyleneZ′, C₂₋₄alkenyleneZ′, C₂₋₄alkynyleneZ and Z′ is CO₂R¹³, the compound of Formula I or I-A formed from the process of the application is further hydrolyzed to provide the free carboxylic acid derivative of the compound of Formula I or I-A. In some embodiments, the compound of Formula I or I-A formed from the process of the application is further hydrolyzed to provide the free carboxylic acid derivative of the compound of Formula I or I-A without purification. Therefore, in some embodiments, when one of R¹, R², R³, R⁴, R⁵ and R⁶ is selected from C₁₋₁₀alkyleneZ′, C₂₋₄alkenyleneZ′, C₂₋₁₀ alkynyleneZ and Z′ is CO₂R¹³, the crude product that comprises the compound of Formula I or I-A is hydrolyzed to provide the free carboxylic acid derivative of the compound of Formula (I) without purification. Accordingly, in some embodiments, the compound of Formula I-1 is not isolated. In some embodiments, when one of R¹, R², R³, R⁴, R⁵ and R⁶ is selected from C₁₋₁₀alkyleneZ′, C₂₋₄alkenyleneZ′, C₂₋₄alkynyleneZ and Z′ is CO₂R¹³, the crude product that comprises the compound of Formula I or I-A is hydrolyzed to provide the free carboxylic acid derivative of the compound of Formula I or I-A without purification in a one pot reaction vessel. Accordingly, in some embodiments, the compound of Formula I-1 is not isolated and is hydrolyzed to provide the free carboxylic acid derivative of the compound of Formula I or I-A in the same vessel used for its preparation to provide the free carboxylic acid derivative of the compound of Formula I or I-A in a one pot, two-step process. In some embodiments, when one of R¹, R², R³, R⁴, R⁵ and R⁶ is selected from C₁₋₁₀ alkyleneZ′, C₂₋₄alkenyleneZ′, C₂₋₄alkynyleneZ and Z′ is CO₂R¹³, the free carboxylic acid derivative of the compound of Formula I or I-A is purified by crystallization. In some embodiments, the free carboxylic acid derivative of the compound of Formula I or I-A is purified without the use of chromatography. Therefore, in some embodiments, the free carboxylic acid derivative of the compound of Formula I or I-A is prepared from the compound of Formula II or II-A and Formula III or III-A without the use of chromatography. In some embodiments, the free carboxylic acid derivative of the compound of Formula I or I-A is purified by trituration. In some embodiments, the free carboxylic acid derivative of the compound of Formula I or I-A is purified by crystallization or trituration using hexane, hexanes, heptane, heptanes, cyclohexane, toluene, and/or xylene and the like. In some embodiments, the free carboxylic acid derivative of the compound of Formula I or I-A is purified by crystallization or trituration using n-hexanes.

In some embodiments, the process provides the free carboxylic acid derivative of the compound of Formula I or I-A in a yield of greater than about 15%, about 20%, about 25%, about 30%, about 35%, about 40%, about 45%, about 50%, about 55%, about 60%, about 65%, about 70%, about 75%, about 80%, about 85%, about 90% or about 95% based on the amount of the compound of Formula II or II-A used in the process of the application. In some embodiments, the process provides the free carboxylic acid derivative of the compound of Formula I or I-A in a yield of greater an about 55%, about 60%, about 65%, about 70%, about 75%, about 80%, about 85%, about 90% or about 95% based on the amount of the compound of Formula II or II-A. In some embodiments, the process provides the free carboxylic acid derivative of the compound of Formula I or I-A in a yield of greater an about 70%, about 75%, about 80%, about 85%, about 90% or about 95% based on the amount of the compound of Formula II or II-A. In some embodiments, the process provides the free carboxylic acid derivative of the compound of Formula I or I-A in a yield of greater than about 60%, greater than about 65%, greater than about 70%, greater than about 75% greater than about 80%, or greater than about 90% based on the amount of the compound of Formula II or II-A.

In some embodiments, the compound of Formula II is the methyl ester of (+)-ABA (II-1):

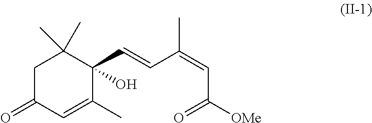 [SMILES: COC(=O)/C=C(C)\C=C\[C@@]1(O)C(C)=CC(=O)CC1(C)C]

and the compound of Formula III is 3-dimethylaminoacrolein (III-1):

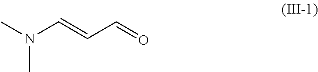

and the process of the application provides the (+)-ABA tetralone methyl ester of Formula I-1:

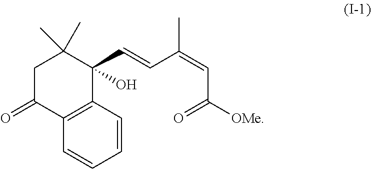 [SMILES: COC(=O)/C=C(C)\C=C\[C@@]1(O)c2ccccc2C(=O)CC1(C)C]

In some embodiments, (+)-ABA tetralone (i.e. (+)-5) is prepared by hydrolyzing the compound of Formula I-1, prepared using the process of the application. Therefore, in some embodiments, the present application includes a two-step process for preparing (+)-ABA tetralone ((+)-5) starting from readily available esters if (+)-ABA such as the methyl ester of (+)-ABA (II-1). In some embodiments, the compound of Formula I-1 is not isolated, and hydrolysis is performed in the same vessel used for its preparation to provide (+)-ABA tetralone (i.e. (+)-5) in a one pot, two-step process. In some embodiments, the overall yield for (+)-ABA tetralone (i.e. (+)-5) in this two-step process is greater than about 60%, greater than about 65%, greater than about 70%, greater than about 75% or greater than about 80%, based on the amount of the methyl ester of (+)-ABA (II-1).

In some embodiments, the compound Formula II is the methyl ester of (+/−)-ABA (11-2) and the compound of Formula III is 3-dimethylaminoacrolein (III-1) and the process of the application provides the (+/−)-ABA tetralone methyl ester of Formula (I-2).

In some embodiments, the compound Formula II is 11-3 and the compound of Formula III is 3-dimethylaminoacrolein (III-1) and the process of the application provides I-3. In some embodiments, the compound of Formula II is the racemate of II-3 and the process of the application provides the racemate of I-3.

In some embodiments, the compound Formula II is II-4 and the compound of Formula III is 3-dimethylaminoacrolein (III-1) and the process of the application provides I-4. In some embodiments, the compound of Formula II is the racemate of II-4 and the process of the application provides the racemate of I-4.

In some embodiments, the application comprises a compound of Formula I or I-A formed by the processes of the application.

A person skilled in the art would appreciate that further manipulation of the substituent groups in the compounds of Formula I or I-A using known chemistry can be performed to provide alternative compounds. For example, salts are formed by methods known to those of ordinary skill in the art, for example, by reacting a compound with an amount of acid or base, such as an equivalent amount, in a medium such as one in which the salt precipitates or in aqueous medium followed by lyophilization.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application.

### Materials and Methods

The act of “concentrating” or to “concentrate” a solution refers to removal of volatiles at water aspirator pressure on a rotary evaporator. Evacuation at ca. 0.1 torr with a vacuum pump generally followed rotary evaporation. Materials were detected on TLC plate by visualization under an ultraviolet lamp (254 nm) and/or by treating the plate with a solution of phosphomolybdic acid (PMA) solution [PMA (40 g), Ce(SO₄)₂ (10 g) and H₂SO₄ (50 mL) and diluted to 1 L with water] followed by charring on a hot plate. Flash column chromatography (FCC) was performed according to Still et al 1978 with Merck Silica Gel 60 (40-63 mm). All mixed solvent eluents are reported as v/v solutions. Unless otherwise noted, all reported compounds were homogeneous by thin layer chromatography (TLC) and by ¹H NMR spectroscopy. The NMR solvent CDCl₃ was passed through small plug of basic alumina prior to use. Unless otherwise noted, NMR spectra were measured in CDCl₃ solution at 500 or 600 MHz (Bruker Avance™) for ¹H and 125 MHz for ¹³C. Signals due to the solvent (¹³C NMR) or residual protonated solvent (¹H NMR) served as the internal standard: CDCl₃ (7.26 δH, 77.23 δC). The ¹H NMR chemical shifts and coupling constants were determined assuming first-order behavior. Multiplicity is indicated by one or more of the following: s (singlet), d (doublet), t (triplet), q (quartet), m (multiplet), br (broad), ap (apparent); the list of couplings constants (J) corresponds to the order of the multiplicity assignment. Coupling constants are reported to the nearest 0.5 Hz (consistent with the digital resolution of ca. 0.2 Hz/pt). The NMR assignments were made based on chemical shifts, coupling constants (J) and multiplicity, and were confirmed by comparing with those previously reported spectra. All other reagents were commercially available and, unless otherwise noted, were used as received.

### Example 1: Preparation of the Compound of Formula I-1 and Free Carboxylic Acid of the Compound of Formula I-1 (i.e. (+)-5) (Milligram Scale)

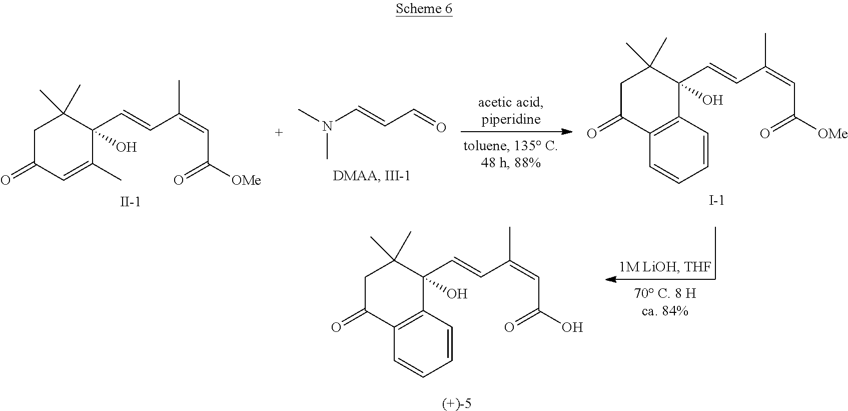

3-Dimethylaminoacrolein (III-1, 0.11 mL, 1.1 mmol) was added to a stirring solution of (+)-ABA methyl ester (II-1, 278 mg, 1.0 mmol) in 5 mL of premade toluene solution (0.12 mL of AcOH and 0.05 mL of piperidine were dissolved in 20 mL of toluene) at room temperature. A Dean-Stark apparatus (filled with the above premade toluene solution) was attached to the flask, which was quickly lowered into a pre-heated oil batch (135° C., oil temp.), and was stirred for 24 h at the same temperature. More 3-dimethylaminoacrolein (0.05 mL, 0.5 mmol), AcOH (0.015 mL) and piperidine (0.007 mL) was added to the reaction mixture and was stirred for another 24 h at 135° C. More 3-dimethylaminoacrolein (0.04 mL, 0.4 mmol), AcOH (0.008 mL) and piperidine (0.004 mL) were added to the reaction mixture and was stirred for another 5 h at 135° C. The reaction mixture was cooled to room temperature, quenched with sat. NaHCO₃ solution and was diluted with water, extracted with EtOAc. The organic layer was separated, washed with brine, dried over Na₂SO₄ and concentrated to obtain the crude as a dark brown syrup. Fractionation of the crude by FCC (20%-30% EtOAc in hexanes) gave (+)-ABA tetralone methyl ester (+)-I-1 (275 mg, 88%) as a pale-yellow syrup. ¹H NMR data closely matched those previously reported (Nyangulu et el, 2006).

The ester (I-1, 275 mg, 0.88 mmol) was dissolved in THF (9 mL), added 1M LiOH solution (9 mL), water (2 mL), and was heated to 70° C. for 16 h. The reaction mixture was cooled to 0° C., diluted with hexane (10 mL), 1M LiOH solution (5 mL), water (5 mL) and the layers separated. The aqueous layer was further washed with diethyl ether (2×20 mL), cooled to 0° C., acidified with 6N HCl solution, and extracted with dichloromethane (3×20 mL). The combined organic layers were dried over Na₂SO₄ and concentrated to obtain the (+)-ABA tetralone (+)-5 (240 mg; 91%, contaminated with an unknown impurity, ca. 7%) as an off-white powder. The ¹H NMR spectrum of the product was identical to the reported spectrum (Nyangulu et al 2006).

### Example 2: Preparation of the Exemplary Compound of Formula I, I-1 and Free Carboxylic Acid of I-A (i.e. (+)-5) (One-Pot, Gram Scale)

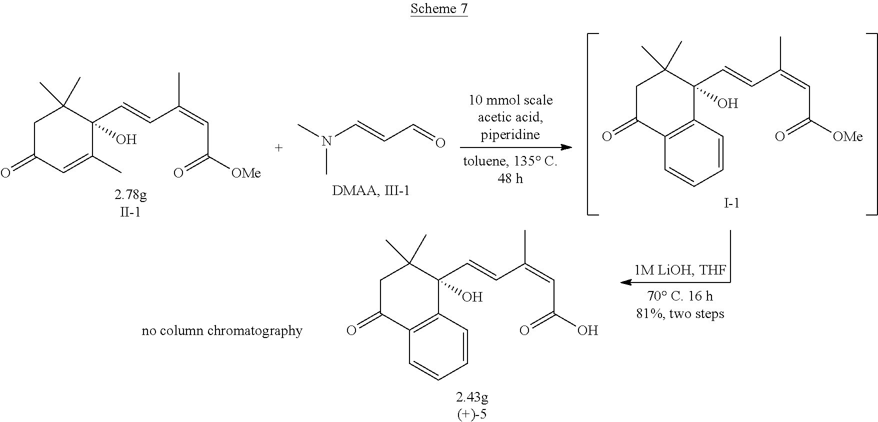

3-Dimethylaminoacrolein (III-1, 1.1 mL, 11.0 mmol) was added to a stirring solution of (+)-ABA methyl ester (11-1, 2.78 g, 10.0 mmol) in 50 mL of premade toluene solution (0.38 mL of AcOH and 0.18 mL of piperidine were dissolved in 80 mL of toluene) at room temperature. A Dean-Stark apparatus (filled with the above premade toluene solution) was attached to the flask, which was quickly lowered into a preheated oil batch (135° C., oil temp.), and was stirred for 16 h at the same temperature. More 3-dimethylaminoacrolein (0.5 mL, 5.0 mmol), AcOH (0.09 mL) and piperidine (0.04 mL) was added to the reaction mixture and was stirred for another 24 h at 135° C. More 3-dimethylaminoacrolein (0.4 mL, 4.0 mmol), AcOH (0.06 mL) and piperidine (0.03 mL) was added to the reaction mixture and was stirred for another 8 h at 135° C. The reaction mixture was cooled to room temperature, quenched with sat. NaHCO₃ solution and was diluted with water, extracted with EtOAc. The organic layer was separated, washed with brine, dried over Na₂SO₄ and concentrated to obtain the crude as a dark brown syrup, which was used in the next reaction without further purification. The crude ester (I-1) was dissolved in THF (90 mL), added 1M LiOH solution (90 mL), water (20 mL), and was heated to 70° C. for 16 h. The reaction mixture was cooled to 0° C., diluted with hexane (100 mL), 1M LiOH solution (50 mL), water (50 mL) and the layers were separated. The aqueous layer was further washed with diethyl ether (2×100 mL), cooled to 0° C., acidified with 6N HCl solution, and extracted with dichloromethane (3×100 mL). The combined organic layers were dried over Na₂SO₄ and concentrated to obtain the crude product as a pale yellow foamy solid (contaminated with ca. 8% unknown impurity). The crude product was triturated with n-hexane (50 mL), filtered, and the solid residue was further triturated twice with 10% EtOAc in hexane (50 mL), filtered, and the solid was dried under vacuum to give the pure ABA tetralone (+)-5 (2.43 g, 81% from (+)-II-1) as a pale brown powder.

¹H NMR (CDCl₃): δ 8.06 (1H, dd, J=7.8, 1.2 Hz, ArH-5′), 7.78 (1H, d, J=16.0 Hz, H-4), 7.62-7.55 (2H, m, ArH-7′ & H-8′), 7.45-7.41 (1H, m, ArH-6′), 6.43 (1H, d, J=16.0 Hz, H-5), 5.75 (1H, s, H-2), 2.83 (1H, d, J=17.0 Hz, H-3′), 2.62 (1H, d, J=17.0 Hz, H-3′), 2.04 (3H, s, H-6), 1.1 (3H, s, H-9′/H-10′), 1.08 (3H, s, H-9′/H-10′). UV_{λmax} (MeOH) nm (In ε): 205 (4.4), 250 (4.4). A small amount of the tetralone acid (+)-5 was crystallized from ethyl acetate to obtain melting point; mp 170-173° C.

### Example 2A: Preparation of the Exemplary Compound of Formula I, I-1 and Free Carboxylic Acid of I-A (i.e. (+)-5) (One-Pot, Gram Scale)

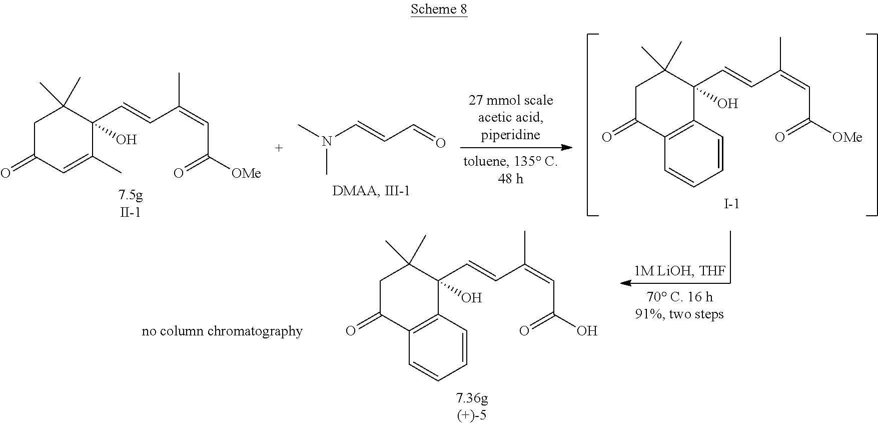

3-Dimethylaminoacrolein (III-1, 3.0 mL, 30.0 mmol) was added to a stirring solution of (+)-ABA methyl ester (11-1, 7.5 g, 27.0 mmol) in 180 mL of premade toluene solution (0.84 mL of AcOH and 0.36 mL of piperidine were dissolved in 180 mL of toluene) at room temperature. A Dean-Stark apparatus (filled with the above premade toluene solution) was attached to the flask, which was quickly lowered into a preheated oil batch (135° C., oil temp.), and was stirred for 24 h at the same temperature (68% conversion was observed by ¹H NMR). More 3-dimethylaminoacrolein (2.4 mL, 24.0 mmol), AcOH (0.20 mL) and piperidine (0.09 mL) were added to the reaction mixture and was stirred for another 24 h at 135° C. (98% conversion was observed by ¹H NMR). The reaction mixture was cooled to room temperature, quenched with sat. NaHCO₃ solution and was diluted with water, extracted with EtOAc. The organic layer was separated, washed with brine, dried over Na₂SO₄ and concentrated to obtain the crude as a dark brown syrup, which was used in the next step without further purification. The crude ester was dissolved in THF (240 mL), added 1M LiOH solution (240 mL), water (50 mL), and was heated to 70° C. for 16 h. The reaction mixture was cooled to 0° C., diluted with diethyl ether (250 mL), 1M LiOH solution (100 mL), water (100 mL) and separated the layers. The aqueous layer was further washed with diethyl ether (250 mL), cooled to 0° C., acidified with 6N HCl solution, and extracted with dichloromethane (3×250 mL). The combined organic layers were dried over Na₂SO₄ and concentrated to obtain the crude as a pale yellow foamy solid. The crude product was triturated with 10% EtOAc in hexane (100 mL), filtered, and the solid was dried under vacuum to give the ABA tetralone (+)-5 (7.36 g, 91% from (+)-II-1) as a pale brown powder. ¹H NMR data for (+)-5 closely matched those previously reported.²

mp:* 170-173° C.; TLC (EtOAc:Hexane:AcOH, 40:55:5 v/v): R_{f}=0.35; ¹H NMR (500 MHz, CDCl₃): δ 8.06 (dd, J=7.8, 1.2 Hz, 1H, ArH-5′), 7.78 (d, J=16.0 Hz, 1H, H-4), 7.62-7.55 (m, 2H, ArH-7′ & H-8′), 7.45-7.41 (m, 1H, ArH-6′), 6.43 (d, J=16.0 Hz, 1H, H-5), 5.75 (s, 1H, H-2), 2.83 (d, J=17.0 Hz, 1H, H-3′), 2.62 (d, J=17.0 Hz, 1H, H-3′), 2.04 (s, 3H, H-6), 1.1 (s, 3H,H-9′/H-10′), 1.08 (s, 3H, H-9′/H-10′); UV_{λmax} (MeOH) nm (log ε): 205 (4.4), 250 (4.4). A small amount of the tetralone acid (+)-5 was crystallized from ethyl acetate to obtain melting point.

### Example 3: Optimization of Reaction Conditions

Optimization studies were performed to improve the yields. All the reactions were performed on 1 mmol scale using Dean-Stark condenser for 24 h unless otherwise noted. The results are shown below in Table 1 with reference to Scheme 9.

Increasing the amount of the aldehyde in the reaction further improved the yield (entry 2). It was observed that the low boiling reagents (AcOH, bp 118° C. & piperidine, bp 106° C.) evaporated from the reaction flask (refluxing at 125-135° C.) and trapped in the Dean Stark apparatus (confirmed by ¹H NMR). While using a directly fitted cold finger or condenser (125° C. oil bath temp., no Dean Stark apparatus) to the reaction flask further improved the yields (entries 3 and 4). Addition of water scavengers (acetic anhydride or trimethyl orthoformate) or excess acid and base resulted in either no reaction or poor yields (entries 5-8). Treatment of II-1(1 equiv) with DMAA (1.8 equiv) in presence of 4:1 ratio of AcOH (0.4 equiv) and piperidine (0.1 equiv) in toluene at 135° C. (Dean-Stark) afforded the desired I-1 in 83% yield by ¹H NMR (entry 9). A reaction of (+)-ABA (1) with DMAA using the established conditions was attempted in a hope to obtain the desired tetralone (+)-5 in a single step; however, this reaction did not afford the product (Table 1, entry 10). Using an alternative acid (propionic acid) and base (2,2,6,6-tetramethylpiperidine) those with higher boiling points (141.2° C. & 152° C., respectively) did not improve the yield (entries 11 and 12); however, the reaction with acetic acid and morpholine (boiling point 129° C.) afforded I-1 in good yield (entry 13). Although, good yields were observed, the starting material II-1 was never consumed completely. Portion wise addition of aldehyde, acid and base to ABA methyl ester (II-1) led the reaction to complete conversion over 48 h (entries 15-17). This is the optimized condition to convert I-1 to I-1 via the cascade reaction. A 10 mmol scale reaction was performed using the optimized conditions to give the crude I-1 (entry 16) which was carried to the hydrolysis step without further purifications. The crude product of I-1 from the 27 mmol scale reaction (entry 17) was carried to the hydrolysis step without further purification.

Saponification of the resulted tetralone ester I-1 produced the tetralone (+)-5 in excellent yield of 91% starting from II-1 (Scheme 9). Notably, no column chromatography was used in either step to obtain pure (>95% by ¹H NMR) tetralone (+)-5.

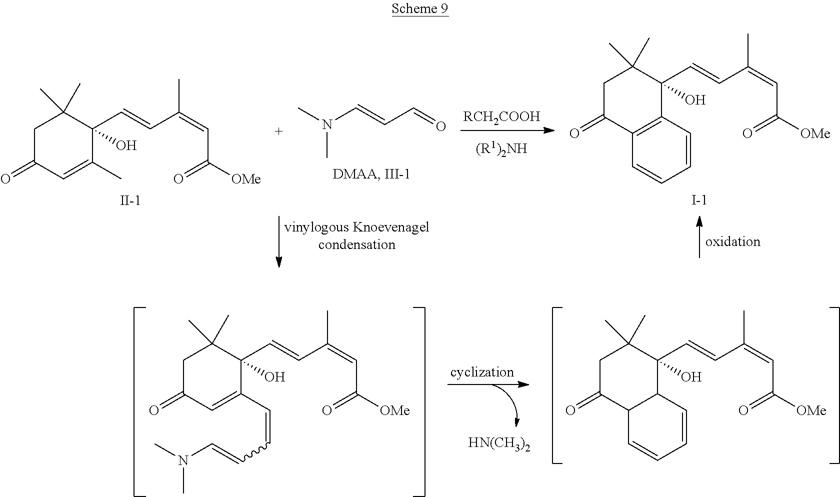

TABLE 1
Optimization study for the cascade reaction.

| | | | | rxn | | | |
|---|---|---|---|---|---|---|---|
| | AcOH | piperidine | DMAA | conc. | Temp. | % | |
| entry | (equiv) | (equiv) | (equiv) | [M] | (° C.) | Yield^{a} | % SM^{a} |
| 1 | 0.2 | 0.05 | 0.9 | 0.5 | 135 | 6 (5) | 94 (91) |
| 2 | 0.2 | 0.05 | 1.8 | 0.2 | 135 | 40 | 60 |
| 3^{b} | 0.2 | 0.05 | 0.9 | 0.5 | 125 | 63 | 36 |
| 4^{b} | 0.4 | 0.1 | 1.8 | 0.2 | 125 | 63 | 26 |
| 5^{b,c} | 0.2 | 0.3 | 1.8 | 0.2 | 125 | 0 | 100 |
| 6^{b,d} | 0.4 | 0.1 | 1.8 | 0.2 | 125 | 25 | 52 |
| 7 | 1.0 | 0.25 | 1.8 | 0.2 | 135 | 50 | 50 |
| 8 | 0.4 | 0 | 1.8 | 0.2 | 135 | 14 | 85 |
| 9 | 0.4 | 0.1 | 1.8 | 0.2 | 135 | 83 (80) | 17 (15) |
| 10^{e} | 0.4 | 0.1 | 1.8 | 0.2 | 135 | 0 | 55 |
| 11 | 0.4^{f} | 0.1 | 1.8 | 0.2 | 135 | 27 | 70 |
| 12 | 0.4^{f} | 0.1^{g} | 1.8 | 0.2 | 135 | 0 | 100 |
| 13 | 0.4 | 0.1^{h} | 1.8 | 0.2 | 135 | 87 | 8 |
| 14 | 0.4 | 0.1 | 2.0 | 0.2 | 135 | 87 | 9 |
| 15ⁱ | 0.4 | 0.1 | 2.0 | 0.2 | 135 | 95 (88) | — |
| 16 ^{i ,j} | 0.4 | 0.1 | 2.0 | 0.2 | 135 | 96 | — |
| 17^{k} | 0.4 | 0.1 | 2.0 | 0.2 | 135 | 98 | — |

All the reactions were performed on 1 mmol scale using Dean-Stark condenser for 24 h unless otherwise noted. ^{a}yield by 1H NMR & isolated yield given in parenthesis. ^{b}cold finger or condenser was used. ° 0.5 equiv of Ac₂O was used as an additive. ^{d}trimethyl orthoformate (excess) was used as an additive. ^{e}(+)-ABA was used. ^{f}propionic acid was used. ^{g}2.2.6.6-tetramethylpiperidine was used. ^{h}morpholine was used. ⁱreagents (acetic acid, piperidine and aldehyde) were added in 3 portions over 48 h reaction time. ^{j}reaction on 10 mmol scale. ^{k}reaction on 27 mmol scale, reagents were added in 2 portions over 48 h.

In summary, an efficient two-step synthesis of enantiopure tetralone ABA (+)-5 from commercially available ABA methyl ester II-1 has been developed, with an overall yield of 91% (no column chromatography). This new synthesis is superior and extremely economical compared to the previously reported synthesis of the precursor racemic tetralone ABA methyl ester I-2, in 6-steps with an overall yield of 15%. This advance now enables the chemical synthesis of enantiopure (+)-5 in large quantities for biological testing in lab and field studies and potential development as a practical plant growth regulator.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference.
Raza, A.; Razzaq, A.; Mehmood, S.; Zou, X.; Zhang, X.; Lv, Y.; Xu, J. Impact of climate change on crops adaptation and strategies to tackle its outcome: A Review. Plants 2019, 8(2), 34.
Helander, J. D. M.; Vaidya, A. S.; Cutler, S. R. Chemical manipulation of plant water use. Bioorganic & Medicinal Chemistry 2016, 24(3), 493-500.
Gupta, M. K.; Lenka, S. K. L.; Rawal, R. K. Agonist, antagonist and signaling modulators of ABA receptor for agronomic and post-harvest management. Plant Physiology and Biochemistry 2020, 148, 10-25.
Cutler, S. R.; Rodriguez, P. L.; Finkelstein, R. R.; Abrams, S. R. Abscisic Acid: Emergence of a Core Signaling Network. Annual Review of Plant Biology 2010, 61, 651-679.
Frackenpohl, J.; Bojack, G.; Baltz, R.; Bickers, U.; Busch, M.; Dittgen, J.; Franke, J.; Freigang, J.; Grill, E.; Gonzalez, S.; Helmke, J.; Hills, M. J.; Hohmann, A.; Von Koskull-Döring, P.; Kleemann, J.; Lange, G.; Lehr, S.; Schmutzler, D.; Schulz, A.; Walther, K.; Willms, L.; Wunschel, C. Potent Analogues of Abscisic Acid—Identifying Cyano-Cyclopropyl Moieties as Promising Replacements for the Cyclohexenone Headgroup. European Journal of Organic Chemistry 2018, 1416-1425.
Yoshida, K.; Kondoh, Y.; Nakano, T.; Bolortuya, B.; Kawabata, S.; Iwahashi, F.; Nagano, E.; Osada, H. New Abscisic Acid Derivatives Revealed Adequate Regulation of Stomatal, Transcriptional, and Developmental Responses to Conquer Drought. ACS Chemical Biology 2021, 16(8), 1566-1575.
Rademacher, W. Plant Growth Regulators: Backgrounds and Uses in Plant Production. Journal of Plant Growth Regulation 2015, 34, 845-872.
Wang, G. T.; Heiman, D. F.; Venburg, G. D.; Nagano, E. Surpin, M. A.; Lustig, J. 3′-substituted-abscisic acid derivatives. WO2016007587A2, 2016.
Abrams, S. R.; Loewen, M. C. In Advances in Botanical Research, Elsevier Inc., 2019; pp 315-339.
Vaidya, A. S.; Peterson, F. C.; Eckhardt, J.; Xing, Z.; Park, S.-Y.; Dejonghe, W.; Takeuchi, J.; Pri-Tal, O.; Faria, J.; Elzinga, D.; Volkman, B. F.; Todoroki, Y.; Mosquna, A.; Okamoto, M.; Cutler, S. R. Click-to-lead design of a picomolar ABA receptor antagonist with potent activity in vivo. Proceedings of the National Academy of Sciences of the United States of America 2021,118, 38.
Nyangulu, J. M.; Nelson, K. M.; Rose, P. A.; Gai, Y.; Loewen, M.; Lougheed, B.; Quail, J. W.; Cutler, A. J.; Abrams, S. R. Synthesis and biological activity of tetralone abscisic acid analogues. Organic & Biomolecular Chemistry 2006, 4, 1400-1412.
Han, X.; Wan, C.; Li, X.; Li, H.; Yang, D.; Du, S.; Xiao, Y.; Qin, Z. Synthesis and bioactivity of 2′,3′-benzoabscisic acid analogs. Bioorganic & Medicinal Chemistry Letters 2015, 25, 2438-2441.
Benson, C. L.; Kepka, M.; Wunschel, C.; Rajagopalan, N.; Nelson, K. M.; Christmann, A.; Abrams, S. R.; Grill, E.; Loewen, M. C. Abscisic acid analogs as chemical probes for dissection of abscisic acid responses in Arabidopsis thaliana. Phytochemistry 2015, 113, 96-107.
Gordon, C. S.; Rajagopalan, N.; Risseeuw, E. P.; Surpin, M.; Ball, F. J.; Barber, C. J.; Buhrow, L. M.; Clark, S. M., Page, J. E.; Todd, C. D.; Abrams, S. R., Loewen, M. C. Characterization of Triticum aestivum Abscisic Acid Receptors and a Possible Role for These in Mediating Fusairum Head Blight Susceptibility in Wheat. Public Library of Science ONE 2016, 11(10).
Wan, C.; Hong, Q.; Zhang, X.; Zeng, Y.; Yang, D.; Che, C.; Ding, S.; Xiao, Y.; Li, J.-Q.; Qin, Z. Role of the Ring Methyl Groups in 2′,3′-Benzoabscisic Acid Analogues. Journal of Agricultural and Food Chemistry 2019, 67(17), 4995-5007.
Abrams, S. R.; Cutler, A. J.; Rose, P.; Nyangulu, J. M.; Nelson, K. M. SYNTHESIS AND BIOLOGICAL ACTIVITY OF BICYCLIC ABA ANALOGS. U.S. Pat. No. 8,969,249B2, 2015.
Shimomura, H.; Etoh, H.; Mizutani, M.; Hirai, N.; Todoroki, Y. Effect of the minor ABA metalolite 7′-hydroxy-ABA on Arabidopsis ABA 8′-hydroxylase CYP707A3. Bioorganic & Medicinal Chemistry Letters 2007, 17, 4977-4981.
Jutz, C.; Wagner, R.-M.; Kraatz, A.; Löbering, H.-G. Umsetzung von vinylogen Formamidiniumsalzen mit Nucleophilen, II. Polymethiniumsalze aus methylenaktiven Ketonen. Justus Liebigs Ann. Chem. 1975, 5, 874-900.
Bredereck, H.; Effenberger, F.; Zeyfang, D. Synthesis and Reactions of Vinylogous Amide Acetals and vinylogous Amidines. Angewandte Chemie, International Edition in English 1965, 4(3), 242.
Bredereck, H.; Effenberger, F.; Zeyfang, D. Hirsch, K.-H. Orthoamide, VII Synthese vinyloger Amidacetale, Aminalester und Amidaminale. Chemische Berichte 1968, 101(12), 4036-4047.
Schröder, L. Preparation of 2-chloropyridine 3-carboxylic acid esters. EP0372654A2, 1990.
W. C. Still, M. Kahn and A. Mitra, Journal of Organic Chemistry 1978, 43, 2923-2925.
Lindsey, B. E.; Rivero, L.; Calhoun, C. S.; Grotewold, E.; Brkljacic, J. Standardized method for high-throughput sterilization of Arabidopsis seeds. Journal of Visualized Experiments 2017, 128, 56587.
Yan, D.; Easwaran, V.; Chau, V.; Okamoto, M.; Ierullo, M.; Kimura, M.; Endo, A.; Yano, R.; Pasha, A.; Gong, Y.; Bi, Y. M.; Provart, N.; Guttman, D.; Krapp, A.; Rothstein, S. J.; Nambara, E. NIN-like protein 8 is a master regulator of nitrate-promoted seed germination in Arabidopsis. Nature Communications 2016, 7, 13179.

1. A process for preparing a compound of Formula (I): 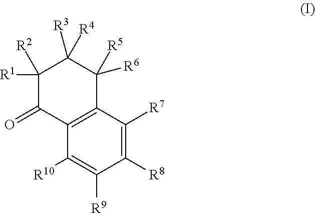
comprising reacting a compound of Formula (II) 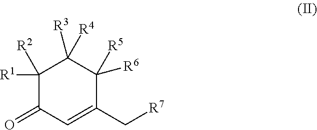
with a compound of Formula (III), 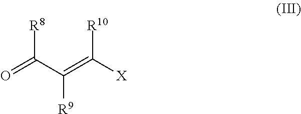
in the presence of a suitable organic acid, suitable organic amine base and a suitable inert solvent, with heating under conditions to remove water,
wherein
R¹, R², R³, R⁴, R⁵ and R⁶ are independently selected from H, halo, OH, CN, C₁₋₃alkyl, C₂₋₄alkenyl, C₂₋₄alkynyl, C₃₋₆cycloalkyl, C₆₋₁₀aryl and C₁₋₃alkyl substituted with one or more OH; wherein each cycloalkyl and aryl optionally substituted with one or more substituents independently selected from OH, halo, C₁₋₆ alkyl and OC₁₋₆alkyl;
one of R¹, R², R³, R⁴, R⁵ and R⁶ is further optionally selected from C₃₋₁₀alkyl, C₄₋₁₀alkenyl, C₄₋₁₀alkynyl, ZC₁₋₁₀alkylene, ZC₂₋₁₀alkenylene, ZC₂₋₁₀alkynylene, C₁₋₁₀alkyleneZ′, C₂₋₁₀alkenyleneZ′, and C₂₋₁₀alkynyleneZ′, wherein each alkyl, alkenyl, alkynyl, alkylene, alkenylene and alkynylene is optionally substituted with one or more substituents independently selected from OH, halo, C₁₋₆alkyl and OC₁₋₆alkyl;
each Z is independently selected from O, S, C(O), S(O), SO₂, NR¹¹, NR¹¹C(O) and C(O)NR¹¹;
each Z′ is independently selected from OR¹², CO₂R¹³, C(O)R¹², NR¹²R¹⁴, and C(O)NR¹²R¹⁴;
X is selected from halo, OR¹⁵ and NR¹⁵R¹⁶
R⁷ is selected from H, halo, OR¹⁷, C(O)R¹⁷, C₁₋₁₀alkyl and C₁₋₁₀alkyl substituted with one or more OH;
R⁸, R⁹ and R¹⁰ are independently selected from H, C₁₋₁₀alkyl, OC₁₋₁₀alkyl, C₆₋₁₀aryl, and C₆₋₁₀aryl optionally substituted with one or more substituents independently selected from OH, halo, C₁₋₆ alkyl and OC₁₋₆alkyl;
R¹¹, R¹², R¹⁴, R¹⁵, R¹⁶ and R¹⁷ are independently selected from H and C₁₋₁₀alkyl; and
R¹³ is C₁₋₁₀alkyl,
provided at least two of R¹, R², R³ and R⁴ are other than H.

2. (canceled)

3. The process of claim 1, wherein R¹ and R² are both H.

4. (canceled)

5. (canceled)

6. (canceled)

7. The process of claim 1, wherein R³ and R⁴ are independently selected from C₁₋₃alkyl.

8. The process of claim 1, wherein one of R⁵ and R⁶ is selected from H, OH, and C₁₋₃alkyl and the other is selected from H, OH, C₁-3alkyl, C₂₋₄alkenyl, C₂₋₄alkynyl, cyclopropyl and phenyl optionally substituted with one or more of OH, C₁₋₄alkyl and OC₁₋₄alkyl.

9. (canceled)

10. (canceled)

11. (canceled)

12. (canceled)

13. (canceled)

14. (canceled)

15. The process of claim 1, wherein one of R⁵ and R⁶ is further optionally selected from C₂₋₆alkenyleneZ′ optionally substituted with one or two of C₁₋₄alkyl.

16. (canceled)

17. The process of claim 15, wherein Z′ is CO₂R¹³, optionally wherein R¹³ is selected from CH₃, CH₂CH₃, CH₂CH₂CH₃, CH(CH₃)₂, CH(CH₃)CH₂CH₃, and CH(CH₃)₃.

18. (canceled)

19. The process of claim 1, wherein X is NR¹⁵R¹⁶, optionally wherein R¹⁵ and R¹⁶ are independently selected from H, CH₃, CH₂CH₃, and CH(CH₃)₂.

20. (canceled)

21. The process of claim 1, wherein R⁷ is selected from H, F, Br, Cl, OR¹⁷, C(O)R¹⁷, C₁₋₈alkyl and C₁₋₈alkyl substituted with one or more OH, optionally wherein R¹⁷ is selected from H, CH₃, CH₂CH₃, and CH(CH₃)₂.

22. (canceled)

23. The process of claim 1, wherein R⁸, R⁹ and R¹⁰ are independently selected from H, C₁₋₆ alkyl, and OC₁₋₆alkyl.

24. The process of claim 1, wherein the compound of Formula II is the methyl ester of (+)-ABA (II-1): 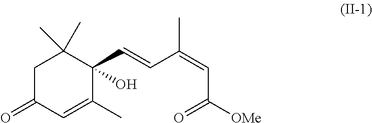 [SMILES: COC(=O)/C=C(C)\C=C\[C@@]1(O)C(C)=CC(=O)CC1(C)C]
and the compound of Formula III is 3-dimethylaminoacrolein (III-1): 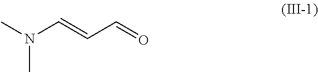
and the process of the application provides the (+)-ABA tetralone methyl ester of Formula I-1:

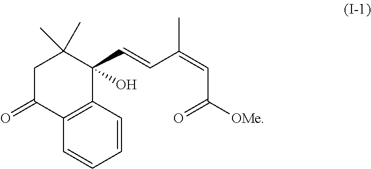 [SMILES: COC(=O)/C=C(C)\C=C\[C@@]1(O)c2ccccc2C(=O)CC1(C)C]

25. The process of claim 1, wherein the process comprises reacting the compound of Formula II with an excess amount of a compound of Formula III, optionally wherein the process comprises reacting the compound of Formula II with about 1.2 to about 3, about 1.2 to about 2.5, about 1.5 to about 2.5, about 1.6 to about 2, or about 1.8 molar equivalents of the compound of Formula III.

26. (canceled)

27. The process of claim 1, wherein the suitable organic acid is a high-boiling point carboxylic acid, optionally wherein the high-boiling point carboxylic acid has a boiling point of greater than about 70° C., greater than about 80° C., greater than about 90° C., greater than about 100° C., greater than about 110° C., greater than about 115° C., greater than about 125° C., greater than about 135° C., greater than about 150° C. or about 160° C.

28. (canceled)

29. (canceled)

30. (canceled)

31. The process of claim 1, wherein the suitable organic amine base is high-boiling point organic amine base, optionally wherein the high-boiling point organic amine base has a boiling point of greater than about 70° C., greater than about 80° C., greater than about 90° C., greater than about 100° C., greater than about 110° C., greater than about 115° C., greater than about 125° C., greater than about 135° C., greater than about 150° C. or about 160° C.

32. (canceled)

33. (canceled)

34. (canceled)

35. (canceled)

36. (canceled)

37. The process of claim 1, wherein the conditions to remove water comprise using a cold finger or condenser, or the conditions to remove water comprise azeotropic distillation conditions.

38. (canceled)

39. The process of claim 1, wherein the suitable inert solvent is a high-boiling-point-solvent having a boiling point of greater than about 70° C., greater than about 80° C., greater than about 90° C., greater than about 100° C., greater than about 110° C., greater than about 115° C., greater than about 125° C., greater than about 135° C., greater than about 150° C. or about 160° C.

40. (canceled)

41. (canceled)

42. (canceled)

43. The process of claim 1, wherein the heating is performed at a temperature of about 75° C. to about 100° C., about 80° C. to about 100° C., about 80° C. to about 90° C. or about 84° C.

44. The process of claim 1, wherein the step of heating is performed at azeotropic reflux temperature or co-distillation temperature of the azeotrope formed between the inert solvent and water formed during the course of the reaction.

45. (canceled)

46. (canceled)

47. (canceled)

48. (canceled)

49. A process for preparing (+)-ABA tetralone (i.e. (+)-5): 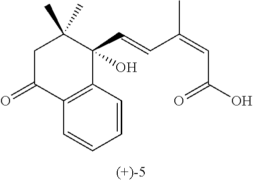
comprising hydrolyzing the compound of Formula I-1, prepared using the process of claim 24.

50. The process of claim 49, wherein the compound of Formula I-1 is not isolated, and hydrolysis is performed in the same vessel.

51. The process of claim 49, wherein (+)-ABA tetralone ((+)-5) is provided in an overall yield of greater than about 60%, greater than about 65%, greater than about 70%, greater than about 75%, greater than about 80%, or greater than 90% based on the amount of methyl ester of (+)-ABA (II-1).
